(19) United States
(12) Patent Application Publication
(10) Pub. No.: US 2024/0374933 A1
(43) Pub. Date: Nov. 14, 2024

(54) TISSUE TREATMENT SYSTEMS AND METHODS WITH ACOUSTIC DOSE MANAGEMENT

(71) Applicant: Otsuka Medical Devices Co., Ltd., Tokyo (JP)

(72) Inventors: Dimitri Augustin, San Mateo, CA (US); Jiayu Chen, Palo Alto, CA (US); Leslie Coleman, Atherton, CA (US); Liang Zhai, Belmont, CA (US); Desmond CHEUNG, San Jose, CA (US); James D. MAZZONE, San Jose, CA (US); Eric DAILEY, San Jose, CA (US); Jaime Merino, Mountain View, CA (US)

(21) Appl. No.: 18/662,757

(22) Filed: May 13, 2024

Related U.S. Application Data
(60) Provisional application No. 63/502,075, filed on May 12, 2023.

(51) Int. Cl. A61N 7/00; A61M 25/10

(52) U.S. Cl. CPC A61N 7/00; A61M 25/1002; A61M 2025/1004; A61M 2025/1047; A61M 2025/1088; A61N 2007/0026; A61N 2007/0043; A61N 2007/0073

(57) ABSTRACT

A tissue treatment system may comprise a catheter including a distal portion on which is located an ultrasound transducer, where at least the distal portion of the catheter is insertable into a segment of a body lumen having a diameter within a range of diameters having a lower subrange and an upper subrange; an excitation source that provides energy to the ultrasound transducer; and a controller that controls the excitation source to cause the ultrasound transducer to emit at least a first amount of acoustic energy and a second amount of acoustic energy greater than the first amount of acoustic energy; where the controller controls the excitation source to cause the ultrasound transducer to emit the first amount of acoustic energy into a body lumen having a diameter within the lower subrange, and the second amount of acoustic energy into a body lumen having a diameter within the upper subrange.

### RELATED APPLICATIONS

This application claims the benefit of priority of U.S. Provisional Patent Application No. 63/502,075 titled TISSUE TREATMENT SYSTEMS AND METHODS WITH ACOUSTIC DOSE MANAGEMENT, filed May 12, 2023, which is incorporated herein by reference in its entirety to provide continuity of disclosure.

### BACKGROUND

This application claims the benefit of priority of U.S. Provisional Patent Application No. 63/502,075, filed on May 12, 2023, which is incorporated herein by reference in its entirety.

### FIELD

This application relates generally to minimally-invasive devices, systems and methods of delivering energy to a targeted anatomical location of a subject, and more specifically, to catheter-based, intraluminal devices and systems configured to deliver ultrasonic energy to treat tissue, such as nerve tissue.

### BACKGROUND INFORMATION

According to the Centers for Disease Control and Prevention (CDC), about one in every three adults suffer from high blood pressure, also known as hypertension. Left untreated, hypertension can result in renal disease, arrhythmias, and heart failure. In recent years, the treatment of hypertension has focused on interventional approaches to inactivate the renal nerves surrounding the renal artery. Autonomic nerves tend to follow blood vessels to the organs that they innervate. Catheters may reach specific structures, such as the renal nerves, that are proximate to the lumens in which the catheters travel. Accordingly, catheter-based systems can deliver energy from within the lumens to inactivate the renal nerves.

### SUMMARY

One example of a tissue treatment system may comprise a catheter including a distal portion on which is located an ultrasound transducer, wherein the catheter is configured such that at least the distal portion of the catheter is insertable into a segment of a body lumen having a diameter within a specified range of diameters, wherein the range of diameters has a lower subrange and an upper subrange; an excitation source configured to selectively provide energy to the ultrasound transducer of the catheter; and a controller communicatively coupled to the excitation source, the controller configured to control the excitation source to cause the ultrasound transducer to cmit two different amounts of acoustic energy, which include a first amount of acoustic energy; and a second amount of acoustic energy, which is greater than the first amount of acoustic energy; wherein the controller is configured to control the excitation source to cause the ultrasound transducer to emit the first amount of acoustic energy into a body lumen having a diameter within the lower subrange, and the second amount of acoustic energy into a body lumen having a diameter within the upper subrange.

One example of a method of operating a tissue treatment system, which may comprise a catheter including a distal portion on which is located an ultrasound transducer, wherein the catheter is configured such that at least the distal portion of the catheter is insertable into a segment of a body lumen having a diameter within a specified range of diameters, wherein the range of diameters has a lower subrange and an upper subrange; an excitation source configured to selectively provide energy to the ultrasound transducer of the catheter; and a controller communicatively coupled to the excitation source, the controller configured to control the excitation source to cause the ultrasound transducer to emit two different amounts of acoustic energy, which include a first amount of acoustic energy and a second amount of acoustic energy, which is greater than the first amount of acoustic energy; may comprise the following steps performed by the controller: controlling the excitation source to cause the ultrasound transducer to selectively emit the first amount of acoustic energy if the diameter of the segment of a body lumen to be treated is within the lower subrange, and to emit the second amount of acoustic energy if the diameter of the segment of the body lumen to be treated is within the upper subrange.

One example of a tissue treatment system may comprise a catheter including a distal portion on which is located an ultrasound transducer, wherein the catheter is configured such that at least the distal portion of the catheter is insertable into a segment of a body lumen having a diameter within a specified range of diameters that is at least 4 mm; an excitation source configured to selectively provide energy to the ultrasound transducer of the catheter; and a controller communicatively coupled to the excitation source, the controller configured to control the excitation source to cause the ultrasound transducer to emit only two different amounts of acoustic energy, which include a first amount of acoustic energy when the diameter of the segment of the body lumen is within a lower subrange of the specified range of diameters; and a second amount of acoustic energy, which is greater than the first amount of acoustic energy, when the diameter of the segment of the body lumen is within an upper subrange of the specified range of diameters.

One example of a method of use of a tissue treatment system having a catheter that includes a distal portion on which is located an ultrasound transducer may comprise inserting the distal portion of the catheter into a segment of body lumen having a diameter within a specified range of diameters that is at least 4 mm, such that the ultrasound transducer is located within the segment of the body lumen having the diameter within the specified range of diameters that is at least 4 mm; and causing the ultrasound transducer to emit about a same amount of acoustic energy when the diameter of the segment of the body lumen is within the specified range of diameters that is at least 4 mm.

One example of a method for use with a tissue treatment system, having a catheter that includes a distal portion on which is located an ultrasound transducer, may comprise inserting the distal portion of the catheter into a segment of a body lumen having a diameter within a specified range of diameters that is at least 4 mm, such that the ultrasound transducer is located within the segment of the body lumen having the diameter within the specified range of diameters that is at least 4 mm; receiving input, at a controller associated with the ultrasound transducer, whether the diameter of the segment of the body lumen, within which the ultrasound transducer is located, is within a lower subrange of the specified range of diameters or an upper subrange of the specified range of diameters; upon receiving input that the diameter of the segment of the body lumen is within the lower subrange of the specified range of diameters, causing the ultrasound transducer to emit a first amount of acoustic energy; and upon receiving input that the diameter of the segment of the body lumen is within the lower subrange of the specified range of diameters, causing the ultrasound transducer to emit a second amount of acoustic energy, which is greater than the first amount of acoustic energy.

One example of a tissue treatment system may comprise a catheter including a distal portion configured to delivery neuromodulation energy to a segment of a body lumen; an excitation source configured to selectively provide neuromodulation energy to the catheter; and a controller communicatively coupled to the excitation source, the controller configured to control the excitation source to cause the catheter to emit about a same amount of neuromodulation energy when a diameter of the segment of the body lumen is within a first specified range of diameters that is at least 4 mm.

### BRIEF DESCRIPTION OF THE DRAWINGS

The novel features of the present disclosure are set forth with particularity in the claims that follow. A better understanding of the features and advantages of the present disclosure will be obtained by reference to the following detailed description that sets forth illustrative embodiments, in which the principles of the present disclosure are utilized, and the accompanying drawings.

FIG. 1 is a perspective view of a tissue treatment system, in accordance with an embodiment.

FIG. 2A is a side view of selected components of the tissue treatment system of FIG. 1, in accordance with an embodiment.

FIG. 2B is a side view of selected components of the tissue treatment system of FIG. 1, in accordance with an embodiment.

FIG. 3 is a perspective view of selected components of the tissue treatment system of FIG. 1 inserted into a body lumen, in accordance with an embodiment.

FIG. 4 is a longitudinal cross-sectional view of a distal region of a tissue treatment system, in accordance with an embodiment.

FIG. 5 is a side view of a tissue treatment system having a compliant balloon inflated to a first inflation diameter, in accordance with an embodiment.

FIG. 6 is a side view of a tissue treatment system having a compliant balloon inflated to a second inflation diameter, in accordance with an embodiment.

FIG. 7 is a diagram of balloon pressure curves of balloons being inflated according to a pressure limiting approach, in accordance with an embodiment.

FIG. 8A illustrates a side view of a balloon having wrinkles when inflated to less than its nominal inflation diameter, in accordance with an embodiment.

FIG. 8B illustrates a perspective view of a balloon having helical folds when inflated to less than its nominal inflation diameter, in accordance with an embodiment.

FIG. 8C illustrates a perspective view of a balloon having longitudinal folds when inflated to less than its nominal inflation diameter, in accordance with an embodiment.

FIG. 8D illustrates a cross-sectional view of a balloon having longitudinal folds when inflated to less than its nominal inflation diameter, in accordance with an embodiment.

FIG. 8E illustrates a perspective view of a balloon having helical folds when inflated to less than its nominal inflation diameter, in accordance with an embodiment.

FIG. 8F illustrates a cross-sectional view of a balloon having helical folds when inflated to less than its nominal inflation diameter, in accordance with an embodiment.

FIG. 9 is a diagram of a balloon pressure curve of a compliant balloon being inflated according to a hybrid inflation approach, in accordance with an embodiment.

FIG. 10 illustrates example details of a fluid supply subsystem, in accordance with an embodiment.

FIG. 11A illustrates example details of a controller, in accordance with an embodiment.

FIG. 11B illustrates example details of the ultrasound excitation source, introduced in FIG. 11A, in accordance with an embodiment.

FIG. 12A is a graph of Acoustic Entry Power versus body lumen size corresponding to an example implementation of single power embodiments for treating target tissue, in accordance with an embodiment.

FIG. 12B is a graph of Acoustic Entry Power versus body lumen size corresponding to another example implementation of the single power embodiments for treating target tissue, in accordance with an embodiment.

FIG. 13A is a graph of Acoustic Entry Power versus body lumen size corresponding to an example implementation of two power embodiments for treating target tissue, in accordance with an embodiment.

FIG. 13B is a graph of Acoustic Entry Power versus body lumen size corresponding to another example implementation of the two power embodiments for treating target tissue.

FIG. 13C is a graph of Acoustic Entry Power versus body lumen size corresponding to a still further example implementation of the two power embodiments for treating target tissue.

FIG. 14 illustrates an example graphical user interface (GUI) that allows a user to specify whether a diameter of a body lumen is within a lower subrange of a specified range of diameters or within an upper subrange of the specified range of diameters, which GUI can be used with the two power embodiments.

FIG. 15 is a high level flow diagram used to summarize of a single power method for use with a tissue treatment system having a catheter that includes a distal portion on which is located an ultrasound transducer.

FIG. 16 is a high level flow diagram used to summarize of a two power method for use with a tissue treatment system having a catheter that includes a distal portion on which is located an ultrasound transducer.

### DETAILED DESCRIPTION

A tissue treatment system, according to certain embodiments, comprises a catheter, an excitation source, and a controller. The catheter includes a distal portion on which is located an ultrasound transducer, where the catheter is configured such that at least the distal portion of the catheter is insertable into a body lumen having a diameter within a specified range of diameters that is at least 4 mm. The excitation source is configured to selectively provide energy to the ultrasound transducer of the catheter. The controller is communicatively coupled to the excitation source.

In some embodiments, the controller of the tissue treatment system is configured to control the excitation source to cause the ultrasound transducer to emit about a same amount of acoustic energy when the diameter of the segment of the body lumen is within the specified range of diameters that is at least 4 mm, e.g., from about 3.0 mm to about 8.0 mm, but not limited thereto.

In other embodiments, the controller of the tissue treatment system is configured to control the excitation source to cause the ultrasound transducer to emit only two different amounts of acoustic energy, which include: a first amount of acoustic energy when the diameter of the segment of the body lumen is within a lower subrange of the specified range of diameters (e.g., 3.0 mm to about 4.9 mm), and a second amount of acoustic energy, which is greater than the first amount of energy, when the diameter of the segment of the body lumen is within an upper subrange of the specified range of diameters (e.g., from about 5.0 mm to about 8.0 mm).

Embodiments are also directed to methods for using the above summarized tissue treatment systems.

A single power method for use with a tissue treatment system having a catheter that includes a distal portion on which is located an ultrasound transducer, comprises inserting the distal portion of the catheter into a segment of body lumen having a diameter within a specified range of diameters that is at least 4 mm, such that the ultrasound transducer is located within the segment of the body lumen having the diameter within the specified range of diameters that is at least 4 mm; and causing the ultrasound transducer to emit about a same amount of acoustic energy when the diameter of the segment of the body lumen is within the specified range of diameters that is at least 4 mm.

A two power method for use with a tissue treatment system having a catheter that includes a distal portion on which is located an ultrasound transducer, comprises inserting the distal portion of the catheter into a segment of a body lumen having a diameter within a specified range of diameters that is at least 4 mm, such that the ultrasound transducer is located within the segment of the body lumen having the diameter within the specified range of diameters that is at least 4 mm. The method also includes determining whether the diameter of the segment of the body lumen, within which the ultrasound transducer is located, is within a lower subrange of the specified range of diameters or an upper subrange of the specified range of diameters. The method further includes causing the ultrasound transducer to emit a first amount of acoustic energy when the diameter of the segment of the body lumen within which the ultrasound transducer is located is determined to be within the lower subrange of the specified range of diameters; and causing the ultrasound transducer to emit a second amount of acoustic energy, which is greater than the first amount of acoustic energy, when the diameter of the segment of the body lumen within which the ultrasound transducer is located is determined to be within the upper subrange of the specified range of diameters.

This description does not include an exhaustive list of all aspects of the present disclosure. It is contemplated that the present disclosure can be practiced from all suitable combinations of the various aspects summarized above, as well as those disclosed in the remainder of this specification and particularly pointed out in the claims. Such combinations have particular advantages not specifically recited in the above summary.

Certain embodiments describe a tissue treatment system having a compliant balloon, and methods of using the tissue treatment system. The tissue treatment system may be an acoustic-based tissue treatment system, e.g., an ultrasound-based tissue treatment system, used to delivery unfocused ultrasonic energy radially outwardly to heat, and thus treat, tissue within a target anatomical region. The unfocused ultrasonic energy may target select nerve tissue within the anatomical region, and may heat such tissue in such a manner as to neuromodulate, e.g., fully or partially ablate, necrose, or stimulate, the nerve tissue. The tissue treatment system can therefore be used to neuromodulate renal nerves to treat hypertension, chronic kidney disease, atrial fibrillation, anxiety, depression, diabetes, sleep apnea, metabolic disorder, insulin resistance, heart failure or other medical conditions. Alternatively, the tissue treatment system may be used in other applications, such as to treat sympathetic nerves of the hepatic plexus within a hepatic artery responsible for blood glucose levels important to treating diabetes and/or sympathetic nerves of the pulmonary artery to treat pulmonary hypertension. In certain embodiments, the tissue treatment catheters are used additionally or alternatively to ablate conductive tissue and/or neuromodulate/ablate sympathetic nerves, such as in the pulmonary vein, to treat atrial fibrillation and/or other cardia arrythmias and/or hypertension. In certain embodiments, the tissue treatment catheters are used additionally or alternatively to treat an autoimmune and/or inflammatory condition, such as rheumatoid arthritis, sepsis, Crohn's disease, ulcerative colitis, and/or gastrointestinal motility disorders by neuromodulating sympathetic nerves within one or more of a splenic artery, celiac trunk, superior or inferior mesenteric artery. Thus, reference to the system as being a renal denervation system, or being used in treating, e.g., neuromodulating, renal nerve tissue is not limiting.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “distal” may indicate a first direction along a longitudinal axis of a tissue treatment system. Similarly, “proximal” may indicate a second direction opposite to the first direction. Such terms are provided to establish relative frames of reference, however, and are not intended to limit the use or orientation of a tissue treatment system to a specific configuration described in the various embodiments below.

A radial access catheter can be less painful to insert, is associated with fewer complications such as bleeding and infection at an access site, and can decrease an overall treatment time. Patients may be discharged on the same day as treatment. The catheter-based system described below can provide a balloon compatible with a guide sheath configured to be inserted via a radial blood vessel of an arm. For example, a balloon of the system 100 can have a crossing-profile that is less than 5 French, less than 0.060 inch (0.1524 cm), and/or less than 0.058 inch (0.14732 cm). Furthermore, the balloon may have a crossing-profile that is 4 French.

The embodiments described herein can provide an ultrasound ablation treatment that is consistently safe and effective. In certain embodiments, a balloon within which is located an ultrasound transducer does not significantly attenuate acoustic energy from the transducer and does not interfere significantly with the acoustic energy emitted by the transducer. For example, in certain embodiments, a balloon is provided consisting of material and a selective thickness such that the balloon has a minimal interference with an energy transmission of a transducer. In other embodiments, a balloon is specifically designed to attenuate acoustic energy emitted from the transducer such that a lower amount of acoustic energy is delivered to tissue being treated when the balloon and transducer are positioned in a relatively small diameter body lumen segment, compared to when the balloon and transducer are positioned in a relatively large diameter body lumen segment. Such embodiments, which utilize the balloon to help deliver appropriate amounts of acoustic energy to tissue being treated, are described in additional detail below.

A tissue treatment system including a catheter having a compliant medical balloon configured for use in a wide range of vessel lumen diameters is provided herein. In an embodiment, the compliant balloon is mounted on a catheter shaft and has an interior containing an ultrasound transducer. The compliant balloon may be formed from a material, and have a structure, that enables the balloon to expand into apposition with a wide range of body lumens. For example, the compliant balloon can be formed from a polyether-based thermoplastic polyurethane, and have a working section that has a predetermined straightness over a range of inflation diameters. The range of inflation diameters can include several diameters that are at least 2 mm different. For example, a first diameter can be in a range of 3.5 to 6 mm, e.g., 5 mm, and a second diameter can be in a range of 8 to 9 mm, e.g., 8.5 mm.

In certain embodiments, a compliant balloon that is arterial limiting is provided. In certain arterial limited embodiments, balloon material is chosen such that the wrinkles (aka folds) in the balloon do not interfere with the sonication. In certain arterial limited embodiments, balloon material is chosen such that the balloon wrinkles (aka folds) in a predictable manner such that the energy profile may be adjusted so that the wrinkles do not interfere with the sonication of the transducer. In other embodiments, compliant balloon material is chosen and the wrinkles (aka folds) are configured to occur in a predictable manner such that when the compliant balloon is partially inflated to less than its nominal balloon diameter, the folded balloon material at least partially attenuates some of the acoustic energy emitted by the transducer, and thereby reduces an amount of the acoustic energy that passes through balloon when a diameter of the body lumen is within a smaller diameter subset of a specified range of diameters, compared to when the diameter of the segment of the body lumen is within a larger diameter subset of the specified range of diameters. More generally, folds in the compliant balloon, which are present when the compliant balloon is partially inflated such that its diameter is less than the nominal balloon diameter of the compliant balloon, are configured to attenuate more of the acoustic energy emitted by the ultrasound transducer, and thereby are configured to allow less of the acoustic energy to pass through the compliant balloon 108 when the compliant balloon is in apposition with a body lumen segment having a diameter that is within a lower diameter subset (e.g., below 5 mm) of the specified range of diameters, compared to when the compliant balloon is in apposition with a body lumen segment that is within a larger diameter subset (e.g., equal to or greater than 5 mm) of the specified range of diameters.

As used herein, an inflation diameter refers to an outer diameter of a cross-sectional shape of the balloon, passing through a center of the transducer. More particularly, a transverse plane oriented orthogonal to a central axis of the balloon can intersect the balloon at an outer profile of the balloon. The outer dimension, e.g., outer diameter, of that profile represents the inflation diameter of the balloon. In an embodiment, the outer diameter can be measured by inflating the balloon, and measuring the outer dimension at the balloon surface radially outward from the transducer. For example, the balloon can be supported and inflated in free space to a given inflation pressure, and a measurement tool, such as a laser caliper, can be used to measure the outer diameter of the inflated balloon. The nominal inflation diameter of a balloon refers the diameter of the balloon when it is inflated to the point that the balloon body is substantially free of wrinkles or other folds and the balloon material has not yet been stretched.

The predetermined straightness of the working section of the balloon can support and center the transducer within a target vessel. In an embodiment, the predetermined straightness includes a cylindricity of the working section being less than a predetermined threshold, e.g., 1 mm. Straightness can be determined with respect to other geometric characterizations, such as a ratio of a radius of curvature of the working section to a length of the compliant balloon, or a ratio of radiuses of curvature of the working section at different inflation diameters. The predetermined straightness of the compliant balloon can compare favorably in terms of tissue contact and transducer support, as compared to typical compliant balloons that tend toward a spherical profile when inflated.

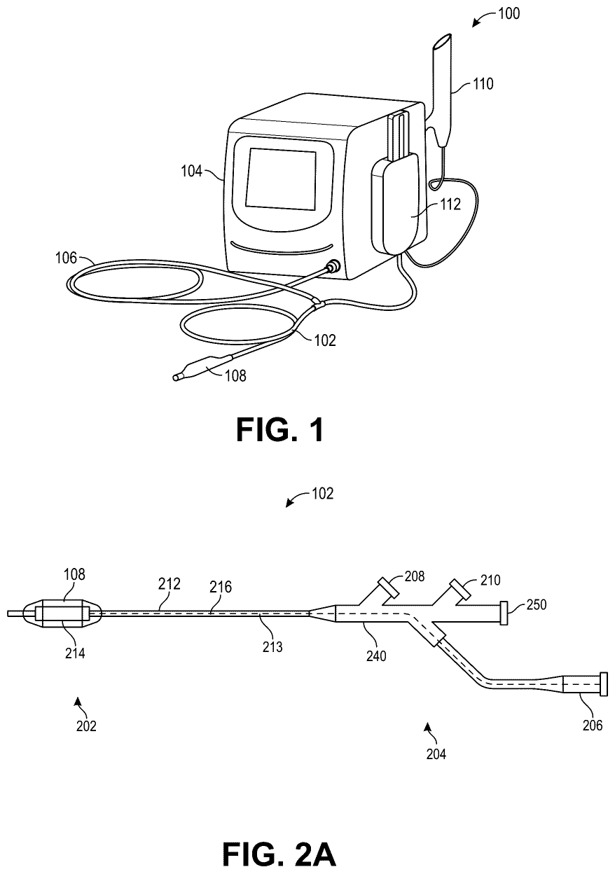

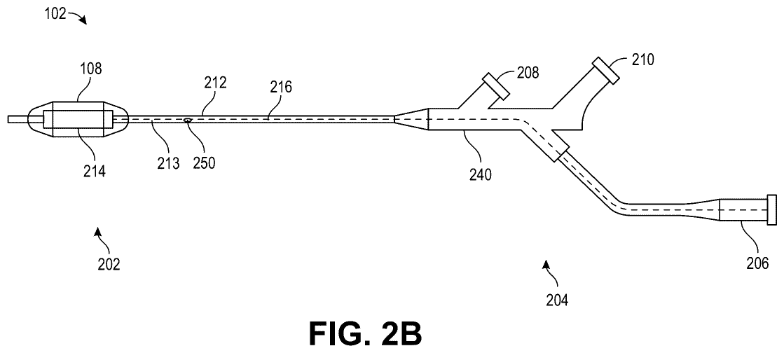

Referring to FIG. 1, a perspective view of selected components of a tissue treatment system is shown in accordance with an embodiment. A tissue treatment system 100 may be a catheter-based system. More particularly, the system 100 can include a catheter 102 that can be delivered intraluminally, e.g., intravascularly, to a target anatomical region of a subject. When so placed, a transducer of the system (FIG. 2A) can be positioned within a target anatomy, e.g., within a body lumen such as a blood vessel. As described below, the transducer can be an ultrasound transducer that may be disposed within a medical balloon 108. The transducer can be activated to deliver unfocused ultrasonic energy radially outwardly so as to suitably heat, and thus treat, tissue within the target anatomical region. The transducer can be activated at a frequency, time, and energy level suitable for treating the targeted tissue.

The tissue treatment system 100 may include the catheter 102, a controller 104, and a connection cable 106. The tissue treatment system 100 further includes a balloon 108, a reservoir 110, a cartridge 112, and a control mechanism, such as a handheld remote control. In certain embodiments, the controller 104 is connected to the catheter 102 through the cartridge 112 and the connection cable 106. In certain embodiments, the controller 104 interfaces with the cartridge 112 to provide cooling fluid to the catheter 102 for inflating and deflating the balloon 108. The controller 104 can also be referred to as the control unit 104.

In an embodiment, a catheter 102 can include a compliant balloon 108 configured to accommodate a range of target vessel sizes, as described below. The compliant balloon 108 can accommodate differences in vessel lumen diameter along the artery length and between left and right renal arteries. For example, the compliant balloon 108 may be configured to treat a blood vessel having a vessel lumen diameter between 3 to 9 mm in diameter. Thus, the compliant balloon 108 can mitigate the need to use several different balloon catheters 102 per procedure. Accordingly, the balloon 108 can reduce procedure times and complexity.

Referring to FIG. 2A, a side view of selected components of the tissue treatment system of FIG. 1 is shown in accordance with an embodiment. The tissue treatment catheter 102 can include a distal region 202 and a proximal region 204. The catheter 102 may have a length that depends on a treatment application. For example, in certain embodiments suitable for, e.g., renal denervation through a femoral access delivery method, the catheter 102 can have a working length (measured from a distal tip of the catheter 102 to a proximal hub 240 of the catheter 102) of 80 to 90 cm, e.g., 85 cm, in the femoral access delivery method. In embodiments suitable for, e.g., renal denervation through a radial access delivery method, the catheter 102 can have a working length of a comparatively longer length. More particularly, the working length can be 150 to 160 cm, e.g., 155 cm. Furthermore, an overall length of the catheter 102 for such application, including a length of cabling extending to an electrical coupling 206, can be longer. More particularly, the cabling can have a length of about 305 cm from the proximal hub 240 to the electrical coupling 206.

The catheter 102 can have a profile that is suitable to accessing a renal artery through the femoral and radial access locations. For example, the catheter 102 may be 4 to 6 French in diameter, e.g., 5 French. The profile is facilitated in part by a catheter shaft 212 having an outer diameter in a range of 0.050 to 0.060 inch, e.g., 0.057 inch.

The distal region 202 of the tissue treatment system 100 may be a portion of the device that is advanced into a target anatomy, e.g., a target vessel having a vessel wall, to treat the target vessel. The distal region 202 can include the balloon 108 mounted on a catheter shaft 212. The balloon 108 can be a compliant balloon having the characteristics described in detail below. For example, the balloon 108 can have a cylindricity that supports and centers a transducer 214 within a range of vessel diameters, and thus, contributes to uniform energy delivery.

The catheter shaft 212 can be an elongated tubular structure that extends longitudinally from a proximal end to a distal end. The balloon 108 can be mounted and supported on the catheter shaft 212 at the distal end. Furthermore, the ultrasound transducer 214 can be mounted on the catheter shaft 212 and contained within the balloon 108. Accordingly, the catheter shaft 212 can facilitate delivery of a cooling fluid to the balloon 108 and delivery of electrical energy to the transducer 214.

The catheter shaft 212 can include one or more lumens (FIG. 4) that may be used as fluid conduits, electrical cabling passageways, guidewire lumens, and/or the like. In an embodiment, the catheter shaft 212 can include a guidewire lumen 213 that is shaped, sized and otherwise configured to receive a guidewire. In an embodiment, the guidewire lumen 213 is an over-the-wire type guidewire lumen, extending from a distal tip of the catheter 102 through an entire length of the catheter shaft 212 to an exit port 250 in the proximal hub 240 of the catheter 102. As described below, the lumen(s) of the catheter shaft 212 may also communicate inflation/cooling fluid from the proximal region 204 to the balloon 108 during balloon expansion.

In an embodiment, a transducer 214 is mounted on the catheter shaft 212 at the distal region 202, within an interior of the balloon 108. The transducer 214 can be an ultrasound transducer 214 used to emit energy toward the vessel wall. For example, the transducer 214 can cmit ultrasound energy circumferentially, e.g., 360 degrees, around the vessel wall. In an embodiment, electric cabling 216 extends from the proximal region 204 to the distal region 202, and is connected to the transducer 214 to generate energy for emission to target tissue.

The ultrasound transducer 214 may include first and second electrodes that are arranged on either side of a cylindrical piezoelectric material, such as lead zirconate titanate (PZT). To energize the transducer 214, a voltage is applied across the first and the second electrodes at frequencies selected to cause the piezoelectric material to resonate, thereby generating vibration energy that is emitted radially outward from the transducer 214. The transducer 214 is designed to provide a generally uniform and predictable emission profile, to inhibit damage to surrounding non-target tissue. In addition, a cooling fluid is circulated through the balloon 108, both prior to, during, and after activation of the transducer 214, so as to reduce heating of an inner lining of the body lumen and to cool the transducer 214. In this manner, the peak temperatures achieved by tissue within the cooling zone remain lower than for tissue located outside the cooling zone.

The proximal region 204 may include one or more connectors or couplings. The connectors or couplings can be electrically connected to the transducer 214 via the electric cabling 216. For example, the proximal region 204 may include one or more electrical coupling 206 that connects to a proximal end of the electric cabling 216. A distal end of the electric cabling 216 can be connected to the transducer 214.

The catheter 102 may be coupled to the controller 104 by connecting the electrical coupling 206 to the connection cable 106. The connection cable 106 may be removably connected to the controller 104 and/or the catheter 102 via a port on the controller 104 and/or the catheter 102. Accordingly, the controller 104 can be used with several catheters 102 during a procedure by disconnecting the coupling of a first catheter, exchanging the first catheter with a second catheter, and connecting a coupling of the second catheter to the controller 104. In certain embodiments, e.g., where only one catheter needs to be used during a procedure, the connection cable 106 may be permanently connected to the controller 104.

In certain embodiments, the proximal region 204 of the catheter 102 may further include one or more fluidic ports. For example, the proximal hub 240 can include a fluidic inlet port 208 and a fluidic outlet port 210, via which an expandable member, e.g., the balloon 108, may be fluidly coupled to the reservoir 110 (FIG. 1). The reservoir 110 can therefore supply cooling fluid to the balloon 108 through the fluidic ports. The reservoir 110 optionally may be included with the controller 104, e.g., attached to the outer housing of the controller 104 as shown in FIG. 1. Alternatively, the reservoir 110 may be provided separately.

Referring to FIG. 2B, a side view of selected components of the tissue treatment system of FIG. 1 is shown in accordance with an embodiment. In an embodiment, the catheter 102 can have a rapid-exchange type guidewire lumen 213. More particularly, the guidewire lumen 213 can extend from the distal tip of the catheter 102 through a partial length of the catheter shaft 212 to an exit port 250 in the distal portion 202 of the catheter 102. For example, a distance from the distal tip to the rapid exchange port 250 may be in a range of 20 to 30 cm, e.g., 23 cm. The proximal hub 240 illustrated in FIG. 2B may differ from the proximal hub 240 illustrated in FIG. 2A, given that the exit port 250 may be moved from the proximal portion 204 to the distal portion 202. Other components of rapid exchange version of the catheter 102 may be similar to those of the over-the-wire version of the catheter 102, and thus, the descriptions of the components illustrated in FIG. 2A can apply to similarly numbered components illustrated in FIG. 2B.

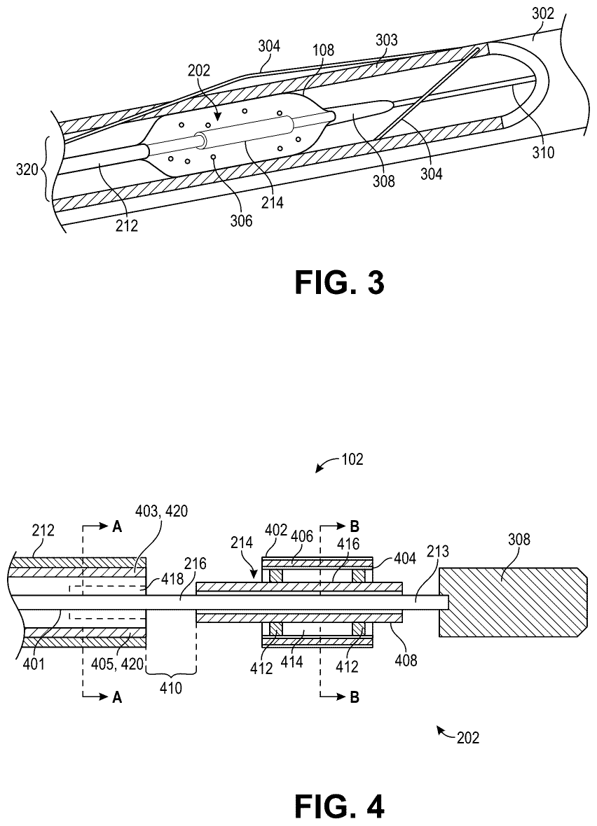

Referring to FIG. 3, a perspective view of additional selected components of the tissue treatment system of FIG. 1 inserted into a body lumen is shown in accordance with an embodiment. The tissue treatment system 100 can be inserted into a body lumen of a subject. For example, a distal region 202 of the catheter 102 of the tissue treatment system 100 can be advanced into a target vessel 302, e.g., a blood vessel such as a renal artery. The target vessel 302 can have a plurality of nerves 304 in an outer layer, e.g., an adventitia layer, of the target vessel 302. In an embodiment, the tissue treatment system 100 includes a guidewire support tip 308 having a lumen that connects to the guidewire lumen 213 of the catheter shaft 212. The support tip 308 can receive the guidewire 310 to allow the device to be tracked over a guidewire 310 to the target anatomy.

When the distal region 202 is disposed in the vessel lumen of the target vessel 302, the transducer 214 and the balloon 108 (or another suitable expandable member) are positioned radially inward from the plurality of nerves 304. The transducer 214 may be disposed partially or completely within the interior of the balloon 108. The balloon 108 can be filled with an inflation fluid 306, e.g., a cooling fluid, to expand the balloon 108. When the balloon 108 is inflated with the inflation fluid 306, the balloon 108 can contact an interior surface, e.g., an intima, of the target vessel. The expanded balloon 108 may therefore have an inflated diameter equal to a lumen diameter 320 of the target vessel 302, and appose the target vessel 302 and center the transducer 214 within the target vessel 302.

In certain embodiments, the transducer 214 may be used to output an acoustic signal when the balloon 108 fully occludes the target lumen. The balloon 108 may center the transducer 214 within the target lumen. In certain embodiments, e.g., suitable for renal denervation, the balloon 108 may be a compliant balloon 108, as described below, which may be inflated in the patient during a procedure at a working pressure of about 1.4 to 2 atm using the inflation fluid 306. The balloon 108 is sized for insertion in the target lumen and, in the case of insertion of the renal artery, for example, the balloon 108 may be selected to have expansion sizes including outer diameters of one or more of 3.5 mm, 4.2 mm, 5 mm, 6 mm, 7 mm, 8 mm, or 9 mm. The balloon 108 may have a burst strength of greater than 45 psi.

In some embodiments, when inflated by being filled with the inflation fluid 306 under the control of the controller 104 within the target vessel 302, a balloon wall of the balloon 108 may be generally parallel with an outer surface of the transducer 214. Optionally, the balloon 108 may be inflated sufficiently as to be in apposition with the target vessel. For example, when inflated, the balloon 108 may at least partially contact, and thus be in apposition with, the inner wall of the target vessel. In other embodiments, the balloon 108 is configured not to contact the target vessel when expanded. The balloon 108 may be maintained at a specified size by pushing fluid into, e.g., via the inlet port 208, and pulling fluid out of, e.g., via the outlet port 210, the balloon 108 at a specified flow rate. More particularly, the inflation fluid 306 can circulate within the balloon 108 to expand the balloon 108.

Referring to FIG. 4, a longitudinal cross-sectional view of the distal region of a tissue treatment system is shown in accordance with an embodiment. In certain embodiments, the catheter shaft 212 may be about 1.8 mm in diameter. As described above, the catheter shaft 212 includes one or more thromboembolism lumens that may be used as fluid conduits, passageways for electrical cabling or the guidewire 310, etc. For example, the catheter shaft 212 may include the guidewire lumen 213 that is shaped, sized and otherwise configured to receive the guidewire 310. The catheter shaft 212 may include a cable lumen 401 (extending through a same shaft as the guidewire lumen 213) for receiving the electrical cabling, and/or fluid lumens for transferring the inflation/cooling fluid, e.g., water, sterile water, saline, 5% dextrose (D5W), other liquids or gases, etc., from and to a fluid source, e.g., the reservoir 110, at the proximal region 204 of the catheter 102 external to the patient. The catheter shaft 212 can include one or more fluid channels 420 to move fluid into or out of a balloon 108. For example, the fluid channel(s) can include an inlet channel 403 to deliver the inflation fluid 306 from the inlet port 208 to the balloon 108 under control of the controller 104. Similarly, the fluid channel(s) can include an outlet channel 405 to remove fluid from the balloon 108 to the outlet port 210. Accordingly, the inlet channel 403 and the outlet channel 405 are in fluid communication with the balloon 108 to circulate fluid through the balloon 108 at a flow rate selected to inflate the balloon 108. The flowrate also controls heat transfer between the balloon 108 and the vessel wall 303 to reduce a likelihood of overheating tissue during treatment. For example, the flowrate can provide for active cooling of about the first millimeter of tissue to preserve the integrity of, e.g., the renal arterial wall.

In certain embodiments suitable for, e.g., renal denervation, the guidewire 310 has a diameter of about 0.36 mm and a length of from about 180 cm to about 300 cm, and is delivered using a 7 French guide catheter 102, having a minimum inner diameter of 2.06 mm and a length less than about 80 cm. In certain embodiments, a 6 French guide catheter 102 is used to deliver the guidewire 310. In certain embodiments, the guide catheter 102 has a length of about 55 cm. In certain embodiments, the guide catheter 102 has a length of about 85 cm and a hemostatic valve is attached to the hub of the guide for continuous irrigation of the guide to decrease the risk of thromboembolism. In certain embodiments, the guidewire lumen 213 is located in the center of the catheter shaft 212 in order to center the transducer 214.

The ultrasound transducer 214 may include a cylindrical tube 402 made of a piezoelectric material, e.g., lead zirconate titanate (PZT), etc., with inner and outer electrodes 404, 406 along the inner and outer surfaces of cylindrical tube 402, respectively. In certain embodiments suitable for, e.g., renal denervation, the piezoelectric material comprises PZT-8 (Navy III). Raw PZT transducers 214 may be plated with layers of copper, nickel and gold to create electrodes on the inner and outer surfaces of the cylinder. Application of alternating current across inner and outer electrodes 406 causes the piezoelectric material to vibrate transverse to the longitudinal direction of the cylindrical tube 402 and radially emit ultrasonic waves.

In addition, the transducer 214 is generally supported via backing member or post 408. In certain embodiments, backing member 408 comprises stainless steel coated with nickel and gold, wherein nickel is used as a bonding material between the stainless steel and gold plating. In certain embodiments suitable for, e.g., renal denervation, the outer diameter of the transducer 214 is about 1.5 mm, the inner diameter is about 1 mm, and the transducer 214 has a length, for example, in a range of 3 to 9 mm, such as 6 mm. The backing member 408 may extend from the distal end of the catheter shaft 212 to the support tip 308. For example, the distal end of the backing member 408 may be positioned within an adjacent opening in the support tip 308, and the proximal end of the backing member 408 may be moveably coupled to the distal end of the catheter shaft 212 via the electrical cabling. In other embodiments, there is a gap 410 between the distal end of the catheter shaft 212 and the backing member 408 supporting the transducer 214, and/or a gap between the backing member 408 and the support tip 308.

In order to permit liquid cooling along both the inner and outer electrodes 406, the backing member 408 may include one or more stand-off assemblies 412. The stand-off assemblies may define one or more annular openings 414 through which cooling fluid may enter the space between the backing member 408 and the inner electrode 404. The backing member 408 may serve as a fluid barrier between the inflation/cooling fluid circulated within the balloon 108 and the lumen of the backing member 408 that receives the guidewire 310. The stand-off assemblies of the backing member 408 may be positioned along each end of the ultrasound transducer 214 (separated by a main post body 416) and couple the cylindrical tube 402 of the ultrasound transducer 214 to the backing member 408. The stand-off assembly 412 may have a plurality of lugs, ribs, or attachment points that engage the inner electrode 404 of the transducer 214. In certain embodiments, the attachment points are soldered to the inner electrode 404 of the transducer 214. The number, dimensions, and placement of the ribs may vary, as desired or required. For example, a total of three ribs are generally equally-spaced apart from one another at an angle of 120 degrees, defining the annular openings 414 through which fluid and blood may enter the interior space of the cylindrical tube 402 between the inner electrode 404 disposed along the inner surface of the cylindrical tube 402 and the backing member 408. In certain embodiments, the maximum outer diameter of the stand-off assemblies is about 1 mm, the outer diameter of the main post body 416 is about 0.76 mm, and the inner diameter of the backing member 408 is about 0.56 mm.

The stand-off assemblies may be electrically conductive, so as to electrically couple the inner electrode 404 of the ultrasound transducer 214 to the backing member 408. One or more conductors of the electrical cabling may be electrically coupled to the backing member 408. Thus, as the controller 104 is activated, current may be delivered from the electrical cabling to the inner electrode 404 of the ultrasound transducer 214 via the backing member 408 and the stand-off assemblies, which advantageously eliminates the need to couple the electrical cabling directly to the inner electrode 404 of the transducer 214.

In an embodiment, the backing member 408 may have an isolation tube (not shown) disposed along its interior surface so as to prevent or reduce the likelihood of electrical conduction between the guidewire 310 and the backing member 408. The isolation tube may be formed of a non-conductive material, e.g., a polymer such as polyimide. The isolation tube may extend from the distal end of the catheter shaft 212 through the lumen of the backing member 408 within the transducer 214 to the support tip 308. The transducer 214 can be mounted on the isolation tube and/or the electrical cabling. In this manner, the transducer 214 can be distally offset from the distal end of catheter shaft 212 by the gap 410.

The catheter 102 may also include a bore 418 extending from the distal end of the catheter shaft 212 proximally within the catheter 102. The bore 418 can be sized and shaped to receive at least a portion of the backing member 408, the electrically insulating isolation tube, and/or the ultrasound transducer 214. Accordingly, during delivery of the catheter 102 to the anatomical region being treated, the backing member 408, the isolation tube, and/or the ultrasound transducer 214 may be retracted within the bore 418 of the catheter 102, e.g., by retracting the electrical cabling, thereby providing sufficient stiffness to the catheter 102 such that the catheter 102 may be delivered in a safe manner.

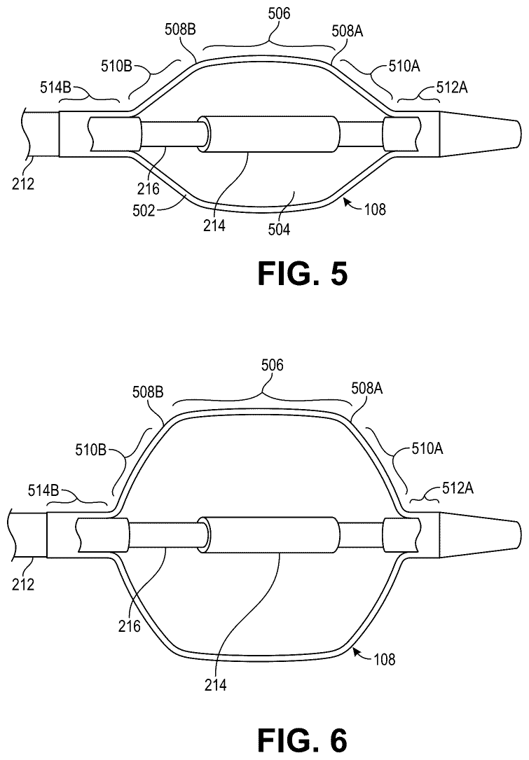

Referring to FIG. 5, a side view of a tissue treatment system having a compliant balloon inflated to a first inflation diameter is shown in accordance with an embodiment. In certain embodiments, the balloon 108 is compliant and configured to be deployed in a wide range of lumen, blood vessel, or artery sizes. For example, the balloon 108 may be capable of adapting to arteries with an inner diameter in the range of 3 mm to 8 mm. Accordingly, using the compliant balloon 108 permits only one catheter 102 to be used during a procedure, advantageously decreasing operating time, e.g., from about 1 hour to about 15 minutes for, e.g., a renal denervation procedure. In certain embodiments, the use of a compliant balloon advantageously decreases the complexity, and thereby the rate of complications, of the procedure.

In certain embodiments, the tissue treatment system 100 is configured to measure the lumen, blood vessel, or artery sizes, and since the balloon 108 is configured to accommodate a wide range of body lumen sizes, e.g., from about 3 mm to about 9 mm renal or accessory arteries, the controller 104 can be programmed to automatically inflate the balloon 108 to the appropriate diameter. Such automation advantageously provides improvements to the complexity of the procedure and mitigates a risk of user error. In certain embodiments, the tissue treatment system 100 having a compliant balloon 108 does not require the user to choose a balloon size and/or switch out catheters to provide multiple sized balloons during a single procedure.

The compliant medical balloon 108 can include a balloon wall 502, which at any longitudinal location, may have a generally annular cross-section. More particularly, the balloon wall 502 can have an outer surface that expands into contact with the target tissue, and an inner surface that defines an interior 504 of the balloon 108. As described above, the transducer 214 can be mounted on the catheter shaft 212, either directly or indirectly (e.g., via the electrical cabling).

The transducer 214 can be positioned within the interior 504 of the balloon 108. More particularly, the balloon 108 can have a balloon body 506, and the balloon body 506 can radially surround the transducer 214. For example, the balloon body 506 can be a generally cylindrical portion of the balloon wall 502 that extends radially around the transducer 214 relative to a longitudinal axis of the catheter shaft 212. The balloon body 506 can extend longitudinally between a plurality of corners 508. For example, a distal corner 508A can define a distal extent of the balloon body 506, and a proximal corner 508B can define a proximal extent of the balloon body 506. In an embodiment, a distance between the corners 508, which defines a length of the balloon body 506, can be equal to or greater than a length of the transducer 214. More particularly, the balloon's body length may be, at a minimum, the length of the transducer 214. Accordingly, the transducer 214 can be positioned such that a proximal end of the transducer 214 is distal to the proximal corner 508B of the balloon 108, and a distal end of the transducer 214 is proximal to the distal corner 508A of the balloon 108. The corners 508 can transition the balloon body 506 into a plurality of shoulders 510. Furthermore, in addition to transitioning the balloon 108 sections, the shape of the corners 508 can have a primary impact on the ability of the balloon 108 to center the transducer 214 within the target vessel 302.

In an embodiment, the plurality of shoulders 510 include a distal shoulder 510A (distal to the balloon body 506) that connects the balloon body 506 to a distal mounting section 512A of the balloon wall 502. Similarly, a proximal shoulder 510B (proximal to the balloon body 506) can connect the balloon body 506 to a proximal mounting section 514B of the balloon wall 502. Accordingly, the shoulders 510 transition the portions of the balloon wall 502 that connect the balloon 108 to the catheter shaft 212 with the portion of the balloon wall 502 that interacts with the target tissue during expansion.

The transducer 214 can be mounted on the isolation tube and/or the backing member 408. In this case the proximal mounting section 514B can be mounted on the catheter shaft 212 proximal to the transducer, but the distal mounting section 514A can be mounted on the transducer, backing member 408 or support tip 308. The mounting sections may be connected to the catheter shaft 212 via thermal, adhesive, or mechanical joints that hermetically seal the balloon 108 to the catheter shaft 212. Accordingly, the interior 504 of the balloon 108, which is between the mounting points, can surround the transducer 214 and provide a space for the inflation/cooling fluid to circulate around the transducer 214 during treatment, as well as prior to and/or after treatment.

It will be appreciated that, as opposed to compliant balloons 108 that primarily function to occlude a target anatomy, the balloon 108 of the tissue treatment system 100 functions to center the transducer 214 within the target vessel 302. The flexibility of the balloon 108 required to achieve the inflation methodologies described below, however, may lead to the transducer 214 becoming eccentric with the vessel lumen if particular features are not implemented in the balloon 108. More particularly, a shape and material of the balloon 108 can be provided as described below to provide a compliant balloon 108 that is also supportive enough to center the transducer 214 within the target vessel 302 during use.

The shape of the balloon 108 can contribute to optimally centering the transducer 214 within the target vessel 302. In an embodiment, the balloon body 506 and the plurality of shoulders 510 meet at round corners 508. The corners 508 may be considered round because, rather than the transition between the shoulder 510 and the balloon body 506 being sharp or angular, the transition has a smooth, arcuate profile. The profile can be described as having a full radius, as opposed to a discrete change in radius that would be apparent, for example, in medical balloons typically used for angioplasty procedures. It has been shown that, as compared to balloon shapes having sharp corners, the round corners 508 of the balloon 108 provide that, when the balloon 108 is inflated within the target vessel 302, the catheter shaft 212 (and the transducer 214 mounted on the catheter shaft 212) remains centered in the target vessel 302.

The material of the balloon 108 can contribute to optimally centering the transducer 214 within the target vessel 302. In certain embodiments, e.g., suitable for renal denervation, the balloon 108 may comprise nylon, polyether block amide (PEBAX®), or other suitable polymers. In an embodiment, the balloon wall 502 is formed from an elastomeric material. For example, the elastomeric material can include a urethane material, such as a thermoplastic polyurethane (TPU). The TPU can be a polyether-based TPU, such as Pellethane®. Alternatively, the balloon wall 502 may be formed from another medical grade polyether-based TPU, such as Isothane®.

Isothane® is a urethane material having a material specification that is closely controlled. As compared to other types of urethane, Isothane® may be particularly useful in that variation in material properties between lots of material are low. More particularly, from lot to lot, Isothane® may have fewer gels and more consistent block chains as compared to other materials. Accordingly, in an embodiment, the raw material used to form the balloon 108 is Isothane®.

A hardness of the balloon material can contribute to the compliance of the balloon 108, e.g., an ability of the balloon to expand and conform to different vessel lumen diameters. The hardness can also contribute to the ability of the balloon 108 to supportively center the transducer 214. Accordingly, the material used to form the balloon wall 502 may have a Shore durometer between about 95A and about 55D. More particularly, the balloon wall material can have a Shore D durometer in a range of 50 to 60. For example, the balloon 108 may be formed from Pellethane® having a Shore D durometer of 55, or Isothane® having a shore durometer of 5095A, 7195A, or 5055D. In a particular embodiment, it has been shown that the balloon wall 502 formed from Isothane® having a Shore D durometer of 55 can provide excellent results in balancing the performance goals of compliant expansion with supportive strength.

Whereas non-compliant balloon inflation is limited by the balloon itself, i.e., the balloon diameter is generally fixed when inflated at different pressures within the expected operating range, and therefore can accommodate a limited range of vessel sizes, compliant balloon expansion can employ multiple methods of inflation that allow the compliant balloon to accommodate a larger range of vessel sizes. The compliant medical balloon 108 of the tissue treatment system 100 described above can be deployed in the target vessel 302 using any of several inflation methodologies. Such methodologies can be termed a “pressure limiting approach,” an “arterial limiting approach,” and a “hybrid approach.”

The pressure limiting approach involves using specific inflation pressures to attain specific balloon diameters to gain apposition to various vessel sizes. The arterial limiting approach involves using a fixed inflation pressure that is used regardless of arterial diameter. The hybrid approach is a combination of the arterial limiting and pressure limiting approaches. The hybrid approach involves using a fixed inflation pressure to gain apposition to smaller arterial diameters, but using alternate (higher) inflation pressures to gain apposition to larger arterial diameters. The strength of the artery effectively determines the size of the balloon 108 at low pressures, and at higher pressures the balloon pressure determines the size of the balloon 108. These inflation paradigms are described in further detail below.

Still referring to FIG. 5, the balloon is shown in a first state and, more particularly, at a first inflation diameter. The inflation diameter can be an outer dimension of the balloon body 506. In an embodiment, the balloon wall 502 has a shape and stiffness (as described herein) such that, when the compliant balloon 108 is inflated to a first inflation pressure of 10 psi, the balloon body 506 of the balloon wall 502 has a cylindrical profile and a first inflation diameter of 3.5 mm to 6 mm. The inflation pressure can correspond to a flowrate of fluid circulated through the interior 504 of the balloon 108 between the inlet channel 403 and the outlet channel 405. For example, the fluid may be circulated at a flowrate of 15 to 35 mL/min (e.g., 25 to 35 mL/min) to inflate the balloon 108 to the inflation pressure of 10 psi, which results in the first inflation diameter of 3 to 6 mm (e.g., 3.5 to 6 mm). The balloon body 506 of the balloon 108 can have the first inflation diameter of 3.5 mm at a first inflation pressure of 10 psi and a flowrate of 30 mL/min. It is noted that the term flowrate can also be equivalently expressed as two words, i.e., flow rate.

In certain embodiments used for the pressure limiting approach, a single balloon 108 can have an inflation diameter that is directly related to the pressure in the balloon 108. More particularly, the outer diameter of the balloon 108 is directly related to the pressure in the balloon 108. According to this embodiment, the higher the pressure, the bigger the balloon 108. It is contemplated that the balloon 108 may have an expansion range of 3.5 to 9 mm. More particularly, the balloon 108 may have a nominal size of 3.5 mm when inflated to the state shown in FIG. 5, however, as the inflation pressure is increased, the inflation diameter may also increase.

Referring to FIG. 6, a side view of a tissue treatment system having a compliant balloon inflated to a second inflation diameter is shown in accordance with an embodiment. When the medical balloon 108 is inflated to a second inflation diameter, e.g., 8 mm, the balloon wall 502 can have essentially the same sections described above. More particularly, the medical balloon 108 can include the mounting sections 512, shoulders 510, and balloon body 506. The corners 508, which transition the balloon body 506 into the shoulders 510, can be rounded. In an embodiment, the arcuate corners 508 can have a same radius as the balloon body 506 and the shoulders 510 such that the balloon wall 502 has a single, arcuate profile of a same radius between the distal mounting section 512 and the proximal mounting section 514. As in FIG. 5, the balloon body 506 can be longer than, and surround, the transducer 214 mounted on the catheter shaft 212. Although the shoulders 510 may be rounded, as shown, the balloon 108 may have angular shoulders instead.

In an embodiment, the balloon wall 502 has a shape and stiffness (as described herein) such that, when the compliant balloon 108 is inflated to a second inflation pressure of 30 psi, the balloon body 506 of the balloon wall 502 has a cylindrical profile and a second inflation diameter of 8 mm to 9 mm. The inflation pressure can correspond to a flowrate of fluid circulated through the interior 504 of the balloon 108 between the inlet channel 403 and the outlet channel 405. For example, the fluid may be circulated at a flowrate of 35 to 50 mL/min (e.g., 40 to 45 mL/min) to inflate the balloon 108 to the inflation pressure of 30 psi, which results in the first inflation diameter of 8 to 9 mm. For example, the balloon body 506 of the balloon 108 can have the second inflation diameter of 8 mm at a second inflation pressure of 30 psi and a flowrate of 40 to 45 mL/min.

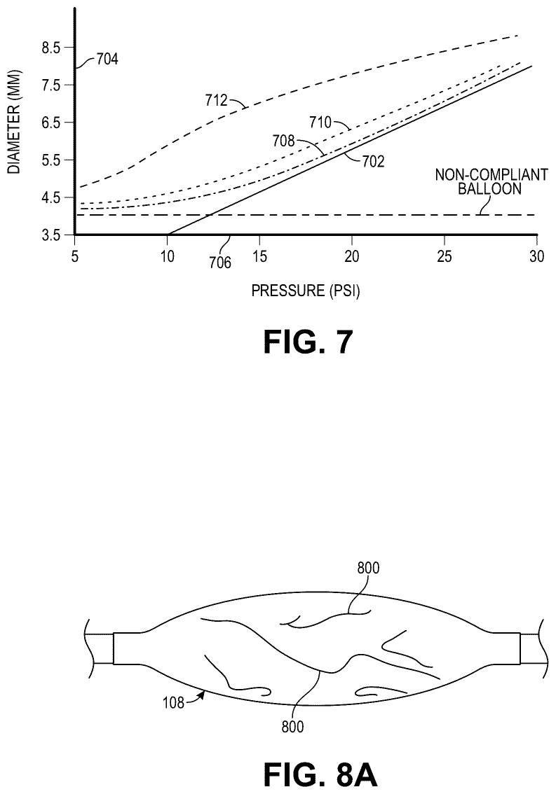

Referring to FIG. 7, a diagram of balloon pressure curves of balloons being inflated according to a pressure limiting approach is shown in accordance with an embodiment. In the pressure limiting approach, the balloon 108 can have a pressure curve that approximates an ideal inflation curve 702. The ideal inflation curve 702 can extend linearly from the first inflation diameter of 3.5 mm at the first inflation pressure of 10 psi to a second inflation diameter of 8 mm at a second inflation pressure of 30 psi. The balloon 108 can therefore accommodate a 3.5 to 8 mm vessel lumen diameter of a same vessel or several vessels. More particularly, an inflation diameter 704 of the balloon 108 corresponds to an inflation pressure 706 of the balloon 108.

The balloon 108 can be inflated by circulating the inflation fluid 306 within the balloon 108. More particularly, circulating the inflation fluid 306 within the balloon 108 generates the inflation pressure that expands the balloon 108 to the inflation diameter. The inflation pressure can be proportional to the flowrate. Accordingly, the inflation fluid 306 can be circulated within the balloon 108 based on a lumen diameter 320 of the target vessel 302 to inflate the balloon 108 to the desired size. For example, the flowrate associated with the second inflation pressure (and the second inflation diameter) may be greater than the flowrate associated with the first inflation pressure (and the first inflation diameter). By way of example, the inflation fluid 306 may be circulated through the balloon 108 at a flowrate between 25 and 45 mL/min to achieve the inflation diameters 704 along the ideal inflation curve 702. In an embodiment, when the inflation fluid 306 is sterile water, the flowrate may be 30 mL/min to achieve the inflation pressure of 10 psi associated with the inflation diameter of 3.5 mm. When the inflation fluid 306 is sterile water, the flowrate may be 40-45 mL/min to achieve the inflation pressure of 30 psi associated with the inflation diameter of 8 mm. In another embodiment, when the inflation fluid 306 is D5W, the flowrate may be 27 mL/min to achieve the inflation pressure of 10 psi associated with the inflation diameter of 3.5 mm. When the inflation fluid 306 is D5W, the flowrate may be 40 mL/min to achieve the inflation pressure of 30 psi associated with the inflation diameter of 8 mm. Accordingly, the pressure limiting approach can utilize flowrates of at least 30 mL/min to achieve inflation pressures of 10-30 psi. It has been shown that a flowrate of 30 mL/min or more circulates fluid sufficiently to adequately cool tissue during renal denervation.

In an embodiment, the balloon 108 approximates the ideal inflation curve 702 over several inflation cycles. For example, the balloon 108 can be inflated to the first inflation diameter (or the second inflation diameter) a first time 708 when the tissue treatment system 100 is introduced into a renal artery. The balloon 108 may be inflated one or more additional times, e.g., a fifth time 710, to treat different regions along a length of the renal artery. It has been shown that, using the materials described above, the inflation curves for the balloon 108 at each inflation cycle approximate each other and the ideal pressure curve. For example, when the balloon 108 is formed from Isothane® 55D, the inflation diameter when the balloon 108 is inflated the first time 708 is within 10% of the inflation diameter when the balloon 108 is inflated the fifth time. By contrast, balloons formed from other materials not contemplated above may exhibit less consistent inflation curves over several cycles. For example, balloons formed from other materials not contemplated above may exhibit inflation diameters 704 at an Nth time 712 that are more than 10% different than inflation diameters 704 at a first time 708. Accordingly, the balloon 108 described herein provides good inflation consistency that permits a single device to be inflated several times to treat a same or different vessels during a single procedure.

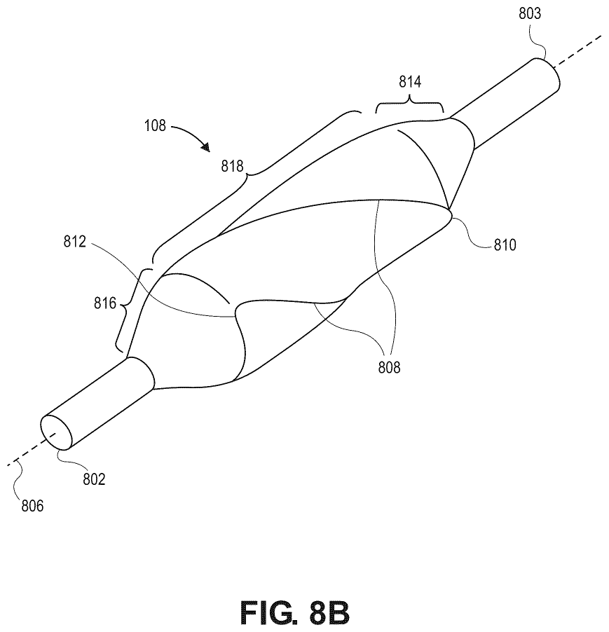

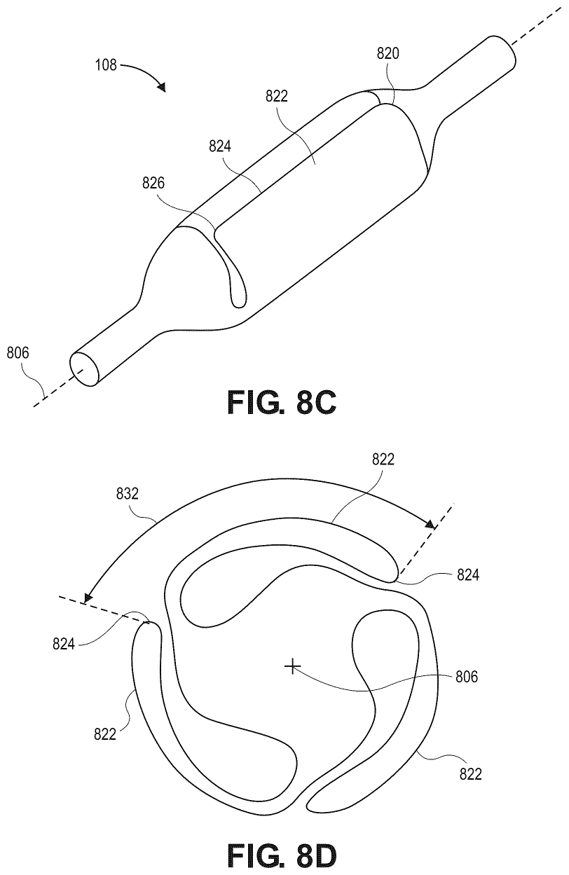

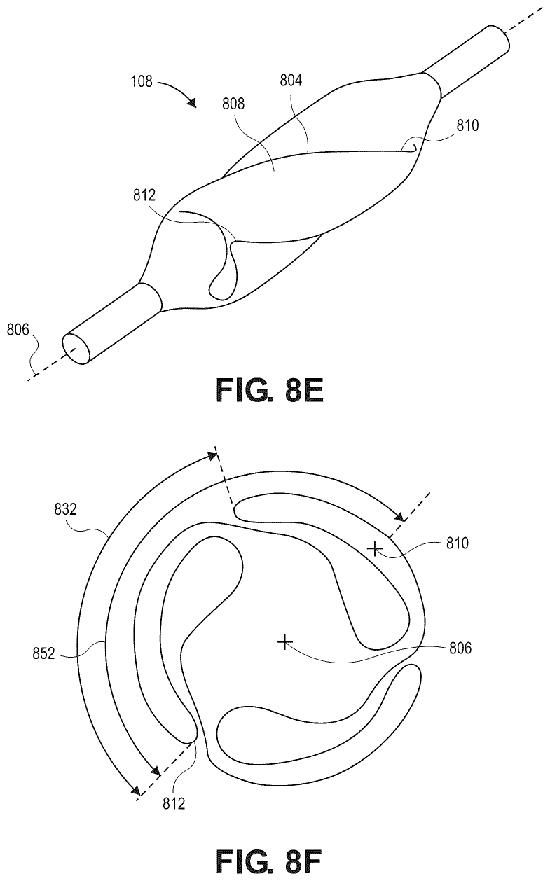

Referring to FIG. 8A, shown therein is a side view of a balloon 108 that includes wrinkles 800 when the balloon 108 is inflated to less than its nominal inflation diameter, which may occur while the balloon is inserted into a target body lumen having a lumen diameter that is less than the nominal inflation diameter. For example, the balloon 108 may have a nominal inflation diameter of 8 mm (or 5 mm, or 6 mm, etc.), yet be inserted into a portion (aka segment) of a body lumen (e.g., a renal artery) having a lumen diameter of 3.5 mm. Therefore, when the balloon 108 is placed in the target body lumen having a smaller lumen diameter than the nominal inflation diameter of the balloon (aka the nominal balloon diameter), the balloon body will contact the body lumen wall before the balloon 108 reaches the nominal inflation diameter (aka the nominal balloon diameter). A hoop strength of the artery, in combination with a low inflation pressure, can therefore keep the balloon 108 at a smaller-than-nominal inflation diameter and can maintain the balloon body in a generally cylindrical profile. For example, the hoop strength of the renal artery and the inflation pressure can prevent the compliant balloon 108 from expanding to the nominal inflation diameter (aka the nominal balloon diameter) of the compliant balloon. In smaller vessels, the balloon 108 may need to be made of excess or thicker material compared to balloons 108 normally intended to accommodate only small body lumens because the balloon 108 must accommodate a wide range of body lumen dimensions, some of which may be beyond the nominal balloon diameter. Accordingly, wrinkles 800 that would otherwise be ironed out due to expansion in larger body lumens can result. More particularly, when the balloon 108 is inflated in the target vessel (aka body lumen) using the arterial limiting approach, the target vessel can constrain the balloon 108, and thus, the balloon 108 can include several wrinkles 800 at the vessel wall where the excess material folds to accommodate the smaller-than-normal diameter. The compliant balloon 108 can be a Pellethane® balloon having a Shore D durometer of 55 and have a double wall thickness of 0.0004 to 0.0014 inch, e.g., 0.0009 inch, and may include several wrinkles 800.

Referring to FIG. 8B, a perspective view of a balloon having helical folds is shown in accordance with an embodiment. The balloon 108 can have a proximal balloon end 802 and a distal balloon end 803. The balloon ends 802, 803 may be ends of cylindrical portions of the balloon 108 that are commonly referred to as legs, necks, or tails. The balloon 108, which may be a compliant balloon or a non-compliant balloon, can have balloon tapered sections 814, 816, commonly referred to as balloon shoulders, extending from the balloon legs 802, 803 to a central, working section 818. The balloon shoulders 814, 816 may be conical, rounded, or have another shape that increases in diameter between a respective balloon leg 802, 803 and the working section 818. The working section 818 can also be referred to as the balloon body 818.

The working section 818, between the balloon shoulders 814 and 816, is shown in a partially inflated state in FIG. 8B. In the partially inflated state (or the deflated state), the balloon 108 includes several helical folds 808 extending about the longitudinal axis 806. More particularly, the helical folds 808 can be present in the balloon wall when the balloon 108 is not fully inflated, e.g., when the balloon has an inflation diameter below a nominal inflation diameter. The helical folds 808 can extend between the proximal balloon end 802 and the distal balloon end 803. More particularly, each helical fold 808 can have a proximal fold end 810 and a distal fold end 812, and the fold can extend between the fold ends in a helical manner, revolving about the longitudinal axis 806.

Referring to FIG. 8C, a perspective view of a balloon having longitudinal folds is shown in accordance with an embodiment. Optionally, the balloon 108 is folded to form several longitudinal folds 822. More particularly, each fold can have a fold edge 824 that extends from the proximal fold end 820 to the distal fold end 826 in a primarily longitudinal direction, parallel to the longitudinal axis 506. The balloon folds can be formed in a wrapping or pleating operation. In the partially inflated state (or the deflated state), the balloon 108 includes several longitudinal folds 822 extending about the longitudinal axis 806. More particularly, the longitudinal folds 822 can be present in the balloon wall when the balloon 108 is not fully inflated, e.g., when the balloon has an inflation diameter below a nominal inflation diameter.

Referring to FIG. 8D, a cross-sectional view of the balloon 108 having longitudinal folds, introduced in FIG. 8C, is shown in accordance with an embodiment. The fold edge 824 can be an apex of the fold at which an outward facing surface of the balloon fold meets an inward facing surface of the balloon 108. More particularly, the fold edge 824 can be a visual edge at which the balloon material folds over on itself. The fold edge 824 may not necessarily be a completely straight line, but may be suggestive of a straight, longitudinal line. The balloon 108 can have several folds, and each fold can have a respective fold edge 824. Furthermore, the fold edges 824 can be separated from each other by a clocking angle 832. The clocking angle 832 can be an angle between a first radial line extending from the longitudinal axis 806 through a first fold edge 824 to a second radial line extending from the longitudinal axis 806 through a second fold edge 824. The clocking angle 832 may be relatively consistent over a length of the balloon 108, between the longitudinal folds 822 and/or between the helical folds 808 described below. For example, the clocking angle 832 can be about 120 degrees in the illustrated cross-section, and may be about 120 degrees between the same two folds at other cross-sections along the length of the balloon 108. The several folds of the balloon 108 may include two or more folds. For example, the several folds can include three folds, as shown in FIG. 8D. Alternatively, the folds (either longitudinal or helical) can include two folds, four folds, etc. The several folds may be evenly distributed such that the clocking angle 832 between each pair of folds is the same. For example, when there are four folds the clocking angle 832 can be 90 degrees, when there are three folds the clocking angle 832 can be 120 degrees, when there are two folds the clocking angle 832 can be 180 degrees, etc. Alternatively, the folds may be unevenly distributed and the clocking angle 832 may vary between different pairs of fold edges 804. For example, the balloon 108 may have three folds with a first pair of fold edges 804 separated by 180 degrees, a second pair of fold edges 804 separated by 90 degrees, and a third pair of fold edges 804 separated by 90 degrees. An embodiment having three folds is described herein. Such example, however, is by no way limiting and it will be appreciated that the balloon 108 may have any number of folds.

Referring to FIG. 8E, a perspective view of a balloon having helical folds is shown in accordance with an embodiment. In an embodiment, the several longitudinal folds 822 can be converted into several helical folds 808. Alternatively, the helical folds 808 may be introduced directly into the balloon 108 without the intermediate operation of forming the longitudinal folds 822. In either case, the distal balloon end 803 can be rotated relative to the proximal balloon end 502 to form several helical folds 808 in the balloon 108. Accordingly, formation of the helical folds 808 can include twisting the balloon 108 to create the folds, or folding the balloon and then twisting the balloon to convert the folds from longitudinal folds 822 into helical folds 808. Twisting the balloon 108 can pre-set the balloon material and reduce a profile of the balloon. In an embodiment, the balloon 108 is twisted by a twist angle that causes the fold edges 804 of the folds to extend helically from the proximal fold end 810 to the distal fold end 812. The folds, therefore, locate the fold edges 804 at predetermined locations around the balloon 108. The folds will remain in these locations when the balloon 108 is inflated and deflated, creating a repeatable distribution of excess balloon material relative to the transducer 214. In the partially inflated state (or the deflated state), the balloon 108 includes several helical folds 808 extending about the longitudinal axis 806. More particularly, the helical folds 808 can be present in the balloon wall when the balloon 108 is not fully inflated, e.g., when the balloon has an inflation diameter below a nominal inflation diameter.

Referring to FIG. 8F, a cross-sectional view of a balloon having helical folds is shown in accordance with an embodiment. The helical folds 808 can have a twist angle 852. The twist angle 852 may be defined by an angular displacement between the proximal fold end 810 and the distal fold end 812. The twist angle 852 can be 90 degrees, 120 degrees, 180 degrees, or any other angle. The twist angle 852 may correspond to the clocking angle 832. The illustrated cross-section can be taken at the distal fold end 812, transverse to the longitudinal axis 806, and the proximal fold end 810 may be hidden, in the view of FIG. 8F, behind an adjacent fold. The angular displacement in the illustrated example can be 180 degrees, resulting in the clocking angle 832 of 120 degrees. Accordingly, the twist angle 852 can be greater than the clocking angle 832. In an embodiment, the twist angle 852 introduces the clocking angle 832, e.g., when the optional operation of forming longitudinal folds 822 is omitted. More particularly, the twisting of the balloon 108 can cause the balloon material to fold over on itself, forming the helical balloon folds. In some cases, the twist angle 852 required to achieve a desired clocking angle 832 may correspond to the clocking angle 832. More particularly, a twist angle 852 of 120 degrees may produce folds having a clocking angle 832 of 120 degrees. Alternatively, the flexibility of the balloon material may require that a larger twist angle 852 be used to achieve the desired clocking angle 832. For example, a twist angle 852 of 180 degrees may be used to produce a clocking angle 832 of 120 degrees. Accordingly, the twist angle 852 can be equal to or greater than the clocking angle 832. For example, the twist angle 852 can be in an angular displacement range including an angle equal to the clocking angle 832 up to an angle equal to five times the clocking angle 832. By way of example, when the clocking angle 832 is 120 degrees, the twist angle 852 can be between 120 degrees to 600 degrees.

The above description is intended to be illustrative and not limiting. More particularly, the balloon 108 may not require a particular clocking angle 832 or twist angle 852 to provide beneficial function in accordance with the principles described herein. The twist angle 852 can be any angle that distributes excess material of the balloon 108 into helical folds 808 and biases the balloon 108 to a same deflated profile and configuration when the balloon is inflated and deflated. Accordingly, the twist angle 852 can introduce repeatable helical folds 508 in the balloon wall, however, the helical folds 808 may have a clocking angle 832 that is independent of the twist angle 852.

In certain embodiments, the balloon 108 is a compliant balloon. The balloon 108 can include an elastomeric material, as described above. The elastomeric material may, for example, include a low durometer (e.g., Shore 80A-55D) urethane material, as described above. Furthermore, the balloon 108 material may be thin. For example, a double wall thickness of the balloon 108 may be 0.0005 inch to 0.0015 inch. The balloon 108 may therefore be highly flexible and the twisting bias can introduce pleats when the thin, elastomeric material folds over onto itself.

When the balloon 108 is rotated to induce a twisting bias in the balloon material, the distal balloon end 803 can be connected to, e.g., attached to, mounted on, etc., a distal tip and/or an isolation tube of the catheter shaft 212. The balloon 108 can be sealed to the catheter shaft 212 such that the interior of the balloon 108 contains the ultrasound transducer 214 and can be filled with a cooling fluid to inflate the balloon. The twisting bias of the balloon 108 in the assembled tissue treatment catheter 102 can be beneficial in creating pre-determined folds in the balloon to facilitate predictable balloon deployment. The pre-determined folds are predictably and repeatably positioned during inflation and deflation to reduce variation in ultrasound therapy as acoustic energy travels through the balloon wall. Furthermore, the folds can provide a thicker balloon wall around the transducer 214 when the balloon 108 is deflated, thereby enhancing protection of the ultrasound transducer.

The term folds, as used herein, is meant to also encompass wrinkles, such as the wrinkles 800 described above with reference to FIG. 8A. Accordingly, when referring to the term folds herein, such folds can, for example, be the wrinkles 800 described above with reference to FIG. 8A, the helical folds 808 described above with reference to FIGS. 8B, 8E and 8F, and/or the longitudinal folds 822 described above with reference to FIGS. 8C and 8D, but are not limited thereto.

In certain embodiments, the balloon 108 can be designed such that folds (e.g., wrinkles) in the balloon do not significantly interfere with energy delivery of the catheter. For example, the folds can have a predictable fold pattern that minimally interferes with energy delivery. More specifically, the predictable fold pattern can have a low density of folds, and/or may have folds that occur in particular locations that are not in the primary energy delivery path.

In other embodiments, the folds (e.g., 800, 808, and/or 822) can have a predictable fold pattern (e.g., wrinkle pattern) that purposefully interferes with acoustic energy delivery, such that the folds attenuate acoustic energy emitted from the transducer 214 within the balloon 108 such that a lower amount of acoustic energy is delivered to tissue being treated when the balloon 108 is in apposition with a body lumen wall of a relatively small diameter body lumen segment, compared to when the balloon 108 is in apposition with a body lumen wall of a relatively large diameter body lumen segment. Such embodiments utilize the balloon 108 to help deliver an appropriate amount of acoustic energy to target tissue being treated. More specifically, a compliant balloon, such as the balloons 108 shown in FIGS. 8A-8F, can be designed so that the folds are configured to occur in a predictable manner such that when the compliant balloon 108 is inserted into a body lumen segment and partially inflated to less than its nominal balloon diameter (such that the balloon is in apposition with a body lumen wall of the body lumen segment), the folded (e.g., wrinkled) balloon surface generates additional acoustic reflections compared to when the balloon 108 is inflated to the point that there are no (or less, and/or smaller) folds (e.g., wrinkles). In such embodiments, the folds cause the acoustic waves to travel a longer propagation path before the acoustic waves exit the balloon 108 and reach the target tissue being treated, whereby this increase in the acoustic propagation path inside the balloon results in acoustic energy loss inside the balloon, prior to the acoustic energy exiting the balloon. Additionally, the folds effectively increase the balloon thickness where folds (e.g., wrinkles) reside. Because the thickness of the balloon material affects how much attenuation is caused by the balloon (i.e., the greater the thickness of the balloon material the greater the attenuation caused), the effective increase in the balloon thickness caused by the folds also contributes to the acoustic energy attenuation. Both of these factors cause a higher percentage of acoustic energy loss before the acoustic energy propagates into the target tissue compared to using a balloon of a same size without folds (e.g., without wrinkles).

More generally, in accordance with certain embodiments, the compliant balloon 108 is designed to at least partially attenuate some of the acoustic energy emitted by the transducer 214 and thereby reduce an amount of the acoustic energy that passes through folded (e.g., wrinkled) balloon material when a diameter of the body lumen segment in which the balloon is inserted is within a smaller diameter subset of a specified range of diameters, compared to when the diameter of the body lumen segment is within a larger diameter subset of the specified range of diameters. For example, less acoustic energy passes through folded (e.g., wrinkled) balloon material when the diameter of the body lumen segment is 3 mm, compared to when the diameter of the body lumen segment is 8 mm. For another example, less acoustic energy passes through folded (e.g., wrinkled) balloon material when the diameter of the body lumen segment is 3 mm, compared to when the diameter of the body lumen segment is 4.9 mm. For still another example, less acoustic energy passes through folded (e.g., wrinkled) balloon material when the diameter of the body lumen segment is 5 mm, compared to when the diameter of the body lumen segment is 8 mm.

The compliant balloons 108 described herein can be inflated using an arterial limiting approach, a pressure limiting approach, or a hybrid thereof. In the arterial limiting approach, the balloon 108 is inflated to a predetermined inflation pressure, regardless of a vessel lumen diameter. More particularly, the low pressure used for the arterial limiting approach can be a fixed pressure that is used regardless of the vessel lumen diameter. For example, the predetermined inflation pressure can be 10 psi or less, and may be used in any target vessel 302 having a vessel lumen diameter less than the nominal inflation diameter of the balloon 108. It will be appreciated that this inflation paradigm is distinct from the pressure limiting approach, which utilizes inflation pressures based on the lumen diameter that is being targeted.

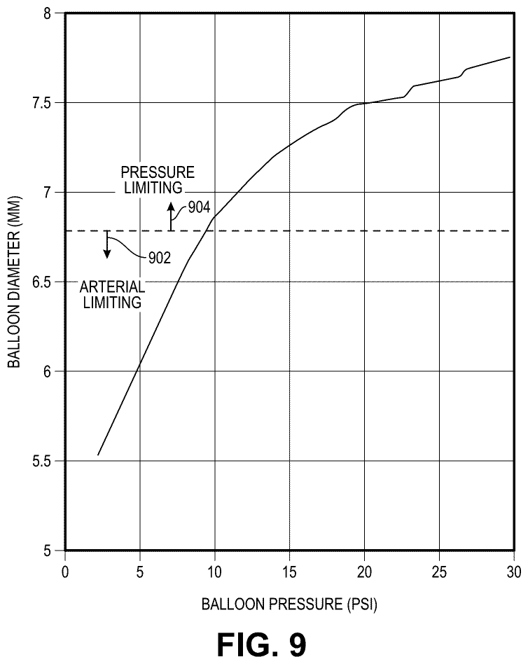

Referring to FIG. 9, a diagram of a balloon pressure curve of a compliant balloon 108 being inflated according to a hybrid inflation approach is shown in accordance with an embodiment. In the hybrid inflation approach, a single, compliant balloon 108 having a nominal inflation diameter can be used to treat vessel lumen diameters smaller than the nominal inflation diameter and larger than the nominal inflation diameter. The compliant balloon 108 can similarly treat the lumen diameters in different vessels, or in different portions of a same vessel, e.g., a distal portion and a proximal portion of the vessel. The balloon 108 may be sized to be at or near a mid-point of a size appropriate for a set of body lumen diameters. For example, with respect to typical renal artery lumen sizes, a balloon 108 having a nominal inflation diameter of 6.75 mm may be provided.

The hybrid approach is a combination of the arterial limiting approach and the pressure limiting approach. In the above example of the balloon 108 having the nominal inflation diameter of 6.75 mm, for an artery less than 6.75 mm, the balloon 108 may be inflated to a low pressure, e.g., 10 psi. Over that inflation range, the balloon 108 may be in an arterial limiting range of operation 902. In the arterial limiting range of operation, the balloon 108 is arterial limited. Accordingly, when the compliant balloon 108 is inflated to a first inflation pressure within a renal artery (or renal artery portion) having a first arterial diameter that is smaller than the nominal inflation diameter of the compliant balloon 108, the hoop strength of the renal artery and the inflation pressure prevents the compliant balloon 108 from expanding to the nominal inflation diameter of the compliant balloon 108.

By contrast, for an artery (or artery portion) larger than the nominal inflation diameter (e.g., 6.75 mm), the pressure in the balloon 108 can be increased to increase the size of the balloon 108. The balloon 108, when operating above the 6.75 mm nominal inflation diameter, can be operating in a pressure limiting range of operation 904. In the pressure limiting range of operation 904, the balloon 108 is pressure limited. Accordingly, when the compliant balloon 108 is inflated to a second inflation pressure higher than the first inflation pressure within a renal artery (or renal artery portion) having a second arterial diameter larger than the nominal inflation diameter of the compliant balloon 108, the second inflation pressure expands the diameter of the compliant balloon 108 to be larger than the nominal inflation diameter of the compliant balloon 108. The inflation pressure can be gradually increased to expand the balloon 108 into apposition with gradually larger arterial diameters. The 6.75 mm nominal inflation diameter is provided by way of example, and as in the embodiments above, the balloon 108 may have a nominal inflation diameter of 3.5 mm, 3.7 mm, 4.5 mm, 5.5 mm, 6.5 mm, or any other diameter that delineates the arterial limiting range of the balloon 108 from the pressure limiting range of the balloon 108.

In an embodiment, the compliant balloon 108 has a nominal inflation diameter of about 4 mm. When the compliant balloon 108 is inflated to a first inflation pressure within a first arterial diameter of a renal artery having a diameter less than 4 mm, the hoop strength of the renal artery and the inflation pressure prevents the compliant balloon 108 from expanding to a diameter larger than the first arterial diameter of the renal artery. When the compliant balloon 108 is inflated to a second inflation pressure higher than the first inflation pressure within a renal artery having a second diameter larger than 4 mm, however, the second inflation pressure expands the diameter of the compliant balloon 108 to be in apposition with the second diameter of the renal artery.

As a further example of the hybrid approach, the compliant balloon 108 can be a Pellethane® balloon having a Shore D durometer of 55 and a nominal inflation diameter of 5.5 mm. The compliant balloon 108 can be inflated at a constant low balloon pressure for apposition in smaller arterial diameters, but for incrementally larger arterial diameters, the pressure is increased to match the balloon size to the artery diameter. Table 1 lists the balloon pressures used to reach the balloon diameter size range. Note that the inflation pressure for diameters up to, and slightly above, the nominal inflation diameter of the compliant balloon are a single, low pressure of 10 psi. The inflation pressures then gradually increase to achieve inflation diameters 704 above 6 mm.

TABLE 1
Balloon Inflation Data

| Balloon Type | Diameter (mm) | Pressure (psi) |
|---|---|---|
| 5.5 mm Pellethane (55D) | ≤6.0 | 10 |
| | 6.1-6.5 | 12 |
| | 6.6-7.0 | 16 |
| | ≥7.0 | 20 |

As described above, the inflation pressure is dependent on the flowrate of the inflation fluid 306 within the balloon 108. Table 2 provides approximate flowrate values of three selected pressures from the complete range of 10 to 20 psi that may be used to inflate the balloon 108 using a hybrid approach. Note that the flowrates are around or above 30 mL/min, which has been shown to effectively cool tissue during renal denervation.

TABLE 2
Balloon Flowrate Data

| Pressure (psi) | Flow Rate (mL/min) |
|---|---|
| 10 | ~24 |
| 15 | ~24 |
| 20 | ~29 |
| 30 | ~40 |

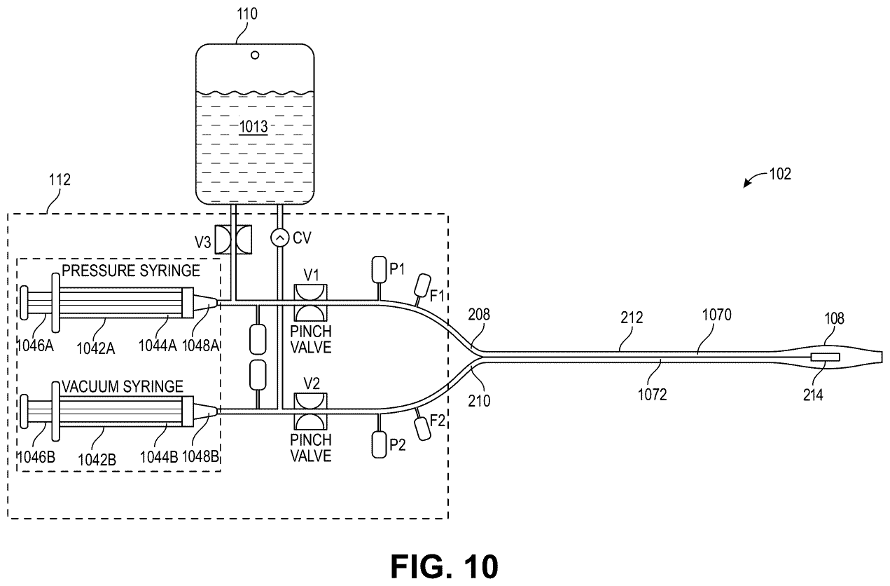

Example details of the cartridge 112 and the reservoir 110, which were introduced above in the above discussion of FIG. 1, will now be described with reference to FIG. 10. The cartridge 112 and/or reservoir 110 can be parts of a fluid supply subsystem. However, it is noted that alternative fluid supply subsystems can alternatively be used to supply cooling fluid to and circulate cooling fluid through the balloon 108, while still being within the scope of the embodiments of the present technology described herein. Referring to FIG. 10, the reservoir 110 is shown as being implemented as a fluid bag, which can be the same or similar to an intravenous (IV) bag in that it can hang from a hook, or the like. The reservoir 110 and the cartridge 112 can be disposable and replaceable items.

The reservoir 110 is fluidically coupled to the cartridge 112 via a pair of fluidic paths, one of which is used as a fluid outlet path (that provides fluid from the reservoir to the cartridge), and the other one of which is used as a fluid inlet path (the returns fluid from the cartridge to the reservoir). The cartridge 112 is shown as including a syringe pump 1040, which includes a pressure syringe 1042a and a vacuum syringe 1042b. The pressure syringe 1042a includes a barrel 1044a, a plunger 1046a, and a hub 1048a. Similarly, the vacuum syringe 1042b includes a barrel 1044b, a plunger 1046b, and a hub 1048b. The hub 1048a, 1048b of each of the syringes 1042a, 1042b is coupled to a respective fluid tube or hose. The cartridge 112 is also shown as including pinch valves V1, V2 and V3, pressure sensors P1, P2, and P3, and a check valve CV. While not specifically shown in FIG. 10, the syringe pump 1040 can include one or more gears and step-motors, and/or the like, which are controlled by the controller 104 (in FIG. 1) to selectively maneuver the plungers 1046 of the pressure syringe 1042a and the vacuum syringe 1042b. Alternatively, the gear(s) and/or step-motor(s) can be implemented within the controller 104, and can be used to control the syringe pump 1040.

In order to at least partially fill the barrel of the pressure syringe 1042a with a portion of the cooling fluid that is stored in the reservoir 110, the pinch valves V1 and V2 are closed, the pinch valve V3 is opened, and the plunger 1046a of the pressure syringe 1042a is pulled upon to draw cooling fluid 1013 into the barrel 1044a of the of the pressure syringe 1042a. The pinch valve V3 is then closed and the pinch valves V1 and V2 are opened, and then the plunger 1046a of the pressure syringe 1042a is pushed upon to expel cooling fluid from the barrel 1044a of the pressure syringe 1042a through the fluid tube attached to the hub 1048a of the pressure syringe 1042a. The cooling fluid expelled from the pressure syringe 1042a enters the fluid lumen 1070 (in the catheter shaft 212), via the fluidic inlet port 208 of the catheter 102, and then enters and at least partially fills the balloon 108. Simultaneously, the plunger 1046b of the vacuum syringe 1042b can be pulled upon to pull or draw cooling fluid from the balloon into the fluid lumen 1072 (in the catheter shaft), through the fluidic outlet port 210 of the catheter 102, and then through fluid tube attached to the hub 1048b of the vacuum syringe 1042b and into the barrel 1044b of the vacuum syringe 1042b. In this manner, the cooling fluid can be circulated through the balloon 108. The balloon 108 can be inflated by supplying more cooling fluid to the balloon than is removed from the balloon. One or more of the pressure sensors P1, P2, and P3 can be used to monitor the pressure in the balloon 108 to achieve a target balloon pressure, e.g., of 10 pounds per square inch (psi), but not limited thereto. Once the balloon is inflated to a target pressure, e.g., between 10 psi and 30 psi, and/or size, the cooling fluid can be circulated through the balloon without increasing or decreasing the amount of fluid within the balloon by causing the same amount of fluid that is removed from the balloon 108 to be the same as the amount of fluid that is provided to the balloon 108. Also, once the target balloon pressure is reached, the ultrasound transducer 214 can be excited to emit ultrasound energy to treat tissue that surrounds the portion of the body lumen (e.g., a portion of a renal artery) in which the balloon 108 and the transducer 214 are inserted. When the ultrasound transducer 214 is emitting ultrasound energy it can also be said that the ultrasound transducer 214 is performing sonication, or that sonication is occurring. During the sonication, cooling fluid should be circulated through the balloon by continuing to push on the plunger 1046a of the pressure syringe 1042a and continuing to pull on the plunger 1046b of the vacuum syringe 1042b.

After the sonication is completed, and the balloon 108 is to be deflated so that the catheter 102 can be removed from the body lumen, the cooling fluid should be returned from the barrel 1044b of the vacuum syringe 1042b to the reservoir 110. In order to return the cooling fluid from the barrel 1044b of the vacuum syringe 1042b to the reservoir 110, the pinch valves V1, V2, and V3 are all closed, and the plunger of the vacuum syringe 1042b is pushed on to expel the cooling fluid out of the barrel of the vacuum syringe 1042b, past the check valve CV, and into the reservoir 110.

The pressure sensors P1, P2, and P3 can be used to monitor the fluidic pressure at various points along the various fluidic paths within the cartridge 112, which pressure measurements can be provided to the controller 104 as feedback that is used for controlling the syringe pump 1040 and/or for other purposes, such as, but not limited to, determining the fluidic pressure within the balloon 108. Additionally, flowrate sensors F1 and F2 can be used, respectively, to monitor the flowrate of the cooling fluid that is being injected (also referred to as a pushed, provided, or supplied) into the balloon 108, and to monitor the flowrate of the cooling fluid that is being drawn (also referred to as a pulled or removed) from the balloon 108. The pressure measurements obtained from the pressure sensors P1, P2, and P3 can be provided to the controller 104 so that the controller 104 can monitor the balloon pressure. Additionally, flowrate measurements obtained from the flowrate sensors F1 and F2 can be provided to the controller 104 so that the controller 104 can monitor the flowrate of cooling fluid being pushed into and pulled from the balloon 108. It would also be possible for one or more pressure sensors and/or flowrate sensors to be located at additional or alternative locations along the fluidic paths that provide cooling fluid to and from the balloon 108.

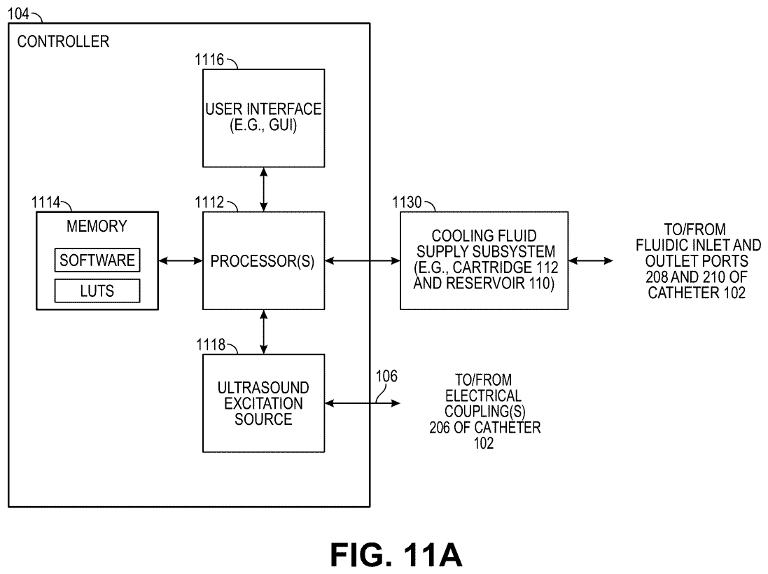

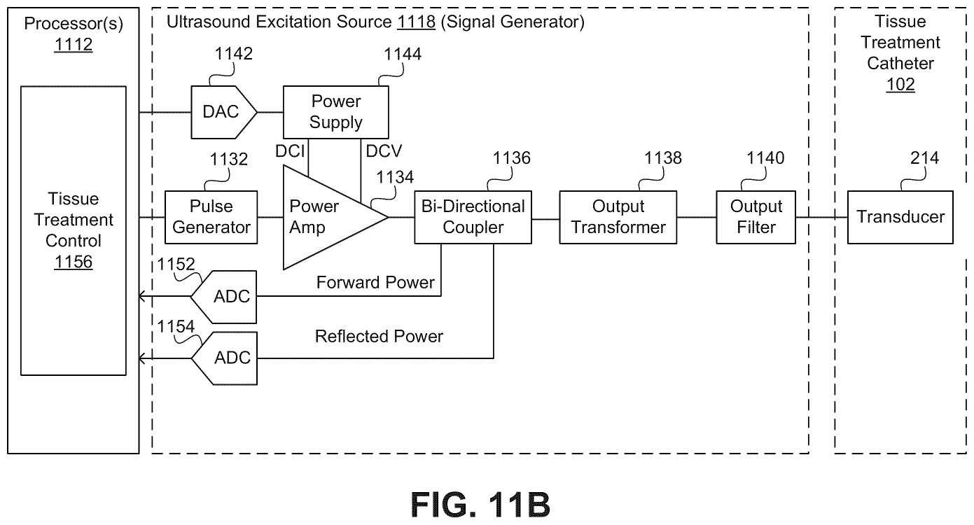

FIG. 11A will now be used to describe an example implementation of the controller 104, which was introduced in FIG. 1. Referring to FIG. 11A, the controller 104 is shown as including one or more processors 1112, a memory 1114, a user interface 1116, and an ultrasound excitation source 1118, but can include additional and/or alternative components. While not specifically shown, a processor 1112 can be located on a control board, or more generally, a printed circuit board (PCB) along with additional circuitry of the controller 104. The processor 1112 can communicate with the memory 1114, which can be a non-transitory computer-readable medium storing instructions. The processor 1112 can execute the instructions to cause the system 100 to perform the methods described herein. The user interface 1116 interacts with the processor 1112 to cause transmission of electrical signals at selected actuation frequencies to the ultrasound transducer 214 via wires of the connection cable 106 and the cabling 282 that extends through the catheter shaft 212. These wires electrically couple the controller 104 to the transducer 214 so that the controller 104 can send electrical signals to the transducer 214, and receive electrical signals from the transducer 214. The processor 1112 can control the ultrasound excitation source 1118 to control the amplitude and timing of the electrical signals so as to control the power level and duration of the ultrasound signals emitted by transducer 214. More generally, the controller 104 can control one or more ultrasound treatment parameters that are used to perform sonication. In certain embodiments, the excitation source 1118 can also detect electrical signals generated by transducer 214 and communicate such signals to the processor 1112 and/or circuitry of a control board. While the ultrasound excitation source 1118 in FIG. 11A is shown as being part of the controller, it is also possible that the ultrasound excitation source 1118 is external to the controller 104 while still being controlled by the controller 104, and more specifically, by the processor 1112 of the controller 104.

The user interface 1116 can include a touch screen and/or buttons, switches, etc., to allow for an operator (user) to enter patient data, select treatment parameters, view records stored on a storage/retrieval unit (not shown), and/or otherwise communicate with the processor 1112. The user interface 1116 can include a voice-activated mechanism to enter patient data or may be able to communicate with additional equipment so that control of the controller 104 is through a separate user interface, such as a wired or wireless remote control. In some embodiments, the user interface 1116 is configured to receive operator-defined inputs, which can include, e.g., a duration of energy delivery, one or more other timing aspects of the energy delivery pulses (e.g., frequency, duty cycle, etc.), power, body lumen length, mode of operation, patient parameter, such as height and weight, and/or verification of artery diameter, or a combination thereof. Example modes of operation can include (but are not limited to): system initiation and set-up, catheter preparation, balloon inflation, verification of balloon apposition, pre-cooling, sonication, post-cooling, balloon deflation, and catheter removal, but are not limited thereto. In certain embodiments, the user interface 1116 provides a graphical user interface (GUI) that instructs a user how to properly operate the system 100. The user interface 1116 can also be used to display treatment data for review and/or download, as well as to allow for software updates, and/or the like.

The controller 104 can also control a cooling fluid supply subsystem 1130, which can include the cartridge 112 and reservoir 110, which were described above with reference to FIGS. 1 and 10, but can include alternative types of fluid pumps, and/or the like. The cooling fluid supply subsystem 1130 is fluidically coupled to one or more fluid lumens (e.g., 327 and 328) within catheter shaft 212 which in turn are fluidically coupled to the balloon 108. The cooling fluid supply subsystem 1130 can be configured to circulate a cooling liquid through the catheter 102 to the transducer 214 in the balloon 108. The cooling fluid supply subsystem 1130 may include elements such as a reservoir 110 for holding the cooling fluid 1013, pumps (e.g., syringes 1042a and 1042b), a refrigerating coil (not shown), or the like for providing a supply of cooling fluid to the interior space of the balloon 108 at a controlled temperature, desirably at or below body temperature. The processor 1112 interfaces with the cooling fluid supply subsystem 1130 to control the flow of cooling fluid into and out of the balloon 108. For example, the processor 1112 can control motor control devices linked to drive motors associated with pumps for controlling the speed of operation of pumps (e.g., syringes 1042a, 1042b). Such motor control devices can be used, for example, where the pumps are positive displacement pumps, such as peristaltic pumps. Alternatively, or additionally, a control circuit may include structures such as controllable valves connected in the fluid circuit for varying resistance of the circuit to fluid flow (not shown). The processor 1112 can monitor pressure measurements obtained by the pressure sensors (e.g., P1, P2 and P3) to monitor and control the cooling fluid through the catheter 102 and the balloon 108. The pressure sensors can also be used to determine if there is a blockage and/or a leak in the catheter 102. While the balloon 108 is in an inflated state, the pressure sensors can be used to maintain a desired pressure in the balloon 108, e.g., at a pressure of between 10 psi and 30 psi, but not limited thereto. As will be described in additional detail below, the processor 1112 can use sensor measurements from one or more of the pressure sensors and/or other sensors to determine when the balloon 108 is in apposition with a body lumen as well as to estimate an inner diameter of a body lumen in order to select an appropriate dose of ultrasound energy to be delivered to treat tissue surrounding the body lumen.

FIG. 11B is used to describe example details of the ultrasound excitation source 1118, according to certain embodiments of the present technology. Where the ultrasound excitation source 1118 is used to produce signals that are used to drive the transducer 214 of the catheter 102, the ultrasound excitation source 1118 can also be referred to as a signal generator 1118. As shown in FIG. 11B, the ultrasound excitation source 1118 (aka signal generator 1118) is communicatively coupled to the processor(s) 1112 of the controller 104. The ultrasound excitation source 1118 (aka signal generator 1118) is shown as including a pulse generator 1132, a power amplifier 1134, a bi-directional coupler 1136, an output transformer 1138, and an output filter 1140. The ultrasound excitation source 1118 (aka signal generator 1118) is also shown as including a digital-to-analog converter (DAC) 1142 and a power supply 1144, which are collectively used to control a gain of the power amplifier 1134. The pulse generator 1132 generates signal pulses under the control of at least one of the processor(s) 1112. More specifically, at least one of the processor(s) 1112, or a tissue treatment control module 1152 thereof, controls the timing and frequency of pulses generated by the pulse generator 1132.

The pulses generated by the pulse generator 1132 are amplified by the power amplifier 1134 to produce amplified pulses that are provided to the output transformer 1138. The output transformer 1138 steps-up the voltage of the pulses output by the power amplifier 1134, and also electrically isolates the circuitry and other components that are downstream of the output transformer 1138, such as the transducer 214, from the circuitry that is upstream of the output transformer 1138, such as the power amplifier 1134 and the pulse generator 1132. For an example, the output transformer 1138 can step-up pulses having a peak-to-peak amplitude of 24V, to pulses having a peak-to-peak amplitude of 60V or 70V, but is not limited thereto. The output filter 1140 shapes the output signal that is provided to the transducer 214, e.g., to convert square wave pulses to sinusoidal pulses, but not limited thereto. The output filter 1140 can also remove noise introduced by the output transformer 1138.

The bi-directional coupler 1136, which is located within signal path between the power amplifier 1134 and the transducer 214, provides a signal indicative of (e.g., proportional to) forward power provided to the transducer 214, and a signal indicative of (e.g., proportional to) reflected power from the transducer 214. In FIG. 11B the bi-directional coupler 1136 is shown as being coupled between the power amplifier 1134 and the output transformer 1138. In alternative embodiments, the bi-directional coupler 1136 can be located downstream of the output transformer 1138, and thus, closer to the transducer 214. The signal indicative of forward power provided to the transducer 214 and the signal indicative of reflected power from the transducer 214, are converted from analog signals to digital signals by analog-to-digital converter (ADC) 1152 and ADC 1154, and are used as feedback signals that are provided to at least one of the processor(s) 1112 to control tissue treatment delivered by the transducer 214. Such feedback is used to accurately control the power provided to the transducer 214.

### Single Power and Two Power Embodiments

In certain catheter-based tissue treatment systems that utilize ultrasound transducers, a user of such a system is required to determine an accurate estimate of a diameter of a body lumen into which a catheter including an ultrasound transducer is to be inserted, so that an appropriate one of numerous (e.g., six) different catheters having an appropriately sized one of numerous (e.g., six) different balloons can be selected for use, and so that an appropriate amount of numerous (e.g., six) different possible amounts of acoustic energy is selected for emission (e.g., in terms of six different power settings). Accordingly, existing catheter-based systems require significant shelf space to stock the portfolio of device sizes. Additionally, the costs of stocking the portfolio of device sizes can be very high. The large product portfolio also creates manufacturing complexities associated with producing a wide range of different device models. Additionally, the need to accurately estimate a diameter of a body lumen increases the time and complexity of a tissue treatment procedure that utilizes such catheter-based tissue treatment systems. The above summarized catheter-based tissue treatment systems can be referred to herein as prior catheter-based tissue treatment systems.

Certain embodiments of the present technology described herein simplify tissue treatment systems and methods (aka procedures) by eliminating the need for a user to accurately estimate a diameter of a segment of body lumen into which a catheter is being inserted, as well as by eliminating the need for multiple catheters to be produced by a manufacturer and purchased and stocked by a medical facility. More specifically, in certain embodiments, about a same amount of acoustic energy is emitted by an ultrasound transducer of the catheter when a diameter of a segment (aka portion) of the body lumen (within which the ultrasound transducer is located) is within the specified range of diameters that is at least 4 mm, or at least 5 mm. In other words, so long as the diameter of the segment of the body lumen (within which the ultrasound transducer is located) is within a specified range of expected diameters, e.g., between about 3 mm and 8 mm, then about a same amount of acoustic energy is emitted by the ultrasound transducer of the catheter to treat target tissue surrounding the body lumen in which the ultrasound transducer of the catheter is located. This significantly simplifies the design of the tissue treatment system that includes a catheter, an excitation source (aka signal generator), and a controller. Additionally, this significantly simplifies the tissue treatment procedure by simplifying what steps need to be performed by a user of the tissue treatment system, in part because the user need not accurately estimate the diameter of the body lumen, and the user need not select from among multiple different catheters to use for the procedure. This also significantly reduces the number of catheters that a manufacturer needs to manufacture, and that a medical facility needs to purchase and stock. The embodiments just summarized can be referred to herein as the single power embodiments.

In an embodiment, about a same amount of acoustic energy is emitted by an ultrasound transducer of the catheter when a diameter of a segment (aka portion) of the body lumen (within which the ultrasound transducer is located) is within the specified range of diameters that is at least 4 mm, or at least 5 mm, while maintaining a total ablation area and depth of ablation to prevent vessel wall damage, in particular damage to the endothelial and medial layers of the vessel wall, while allowing sufficient arterial nerve and/or peri-arterial nerve damage to be therapeutic. A minimum ablation distance from the arterial lumen (e.g., about 0.5 mm to about 1.5 mm) is maintained to preserve the arterial wall, while a maximum ablation distance is maintained to preserve the safety of periarterial organs. For example, a lesion depth of 5 mm-7 mm, e.g., 5.5 mm to 6 mm, may be maintained over the specified range of diameters that is at least 4 mm, or at least 5 mm, or at least 6 mm.

In other embodiments, rather than about a same amount of acoustic energy always being emitted by the ultrasound transducer of the catheter, a first amount of acoustic energy is emitted by the ultrasound transducer when a diameter of the segment of the body lumen (within which the ultrasound transducer is located) is within a lower subrange of a specified range of diameters (that is at least 1 mm, or at least 1.5 mm, or at least 2 mm, or at least 2.5 mm), and a second amount of acoustic energy (which is greater than the first amount of energy) is emitted by the ultrasound transducer when the diameter of the segment of the body lumen (within which the ultrasound transducer is located) is within an upper subrange of the specified range of diameters (that is at least 2.5 mm, or at least 3 mm, or at least 3.5 mm, or at least 4 mm, or at least 4.5 mm). Such embodiments can be referred to herein as the two power embodiments. While not quite as simple as the single power embodiments, the two power embodiments still significantly simplify the design, manufacture and use of a tissue treatment system that includes a catheter, an excitation source, and a controller, compared to the prior catheter-based tissue treatment systems summarized above (where there was a need to determine an accurate estimate of a diameter of a body lumen into which a catheter is to be inserted, so that an appropriate one of numerous (e.g., six) different catheters having an appropriately sized one of numerous (e.g., six) different balloons can be selected for use, and so that an appropriate amount of numerous (e.g., six) different possible amounts of acoustic energy is selected for emission). Advantageously, the two power embodiment provides a total ablation area and depth of ablation that prevents vessel wall damage, in particular damage to the endothelial and medial layers of the vessel wall, while allowing sufficient arterial nerve and/or peri-arterial nerve damage to be therapeutic. A minimum ablation distance from the arterial lumen (e.g., about 0.5 mm to about 1.5 mm) is maintained to preserve the arterial wall, while a maximum ablation distance is maintained to preserve the safety of periarterial organs. For example, a lesion depth of 5 mm-7 mm, e.g., 5.5 mm to 6 mm) may be maintained over the entire range of diameters treated by a two power embodiment. A benefit of the two power embodiment over the one power embodiments is that the two power embodiments should be able to provide safe and effective tissue treatment over a larger range of body lumen diameters than the one power embodiments, because a lower power is used with a smaller diameter subrange of body lumen diameters, and a larger power is used with a larger diameter subrange of body lumen diameters.

A tissue treatment system, of the single power embodiments and two power embodiments, includes a catheter, a controller (aka a control unit), and an excitation source (aka a signal generator). For the following discussion, it is assumed that the tissue treatment system 100 is utilized, which includes the catheter 102, the controller 104, and the excitation source 1118. Further, it is assumed that the catheter includes a distal portion (e.g., the shaft 212) on which is located the ultrasound transducer 214, wherein the catheter is configured such that at least the distal portion of the catheter is insertable into a body lumen (e.g., a renal artery) having a specified range of diameters that is at least 4 mm, or at least 5 mm (e.g., from about 3.0 mm to about 8.0 mm). However, it should be noted that the use of alternative catheters, controllers, excitation sources, and transducers is also possible. The excitation source 1118 is configured to selectively provide energy to the ultrasound transducer 214 of the catheter 102, in response to which the ultrasound transducer emits 214 an acoustic signal having an acoustic frequency, power and duration. The controller 104 is communicatively coupled to the excitation source 1118 and is configured to control the excitation source 1118 to cause the ultrasound transducer 214 to emit about a same amount of acoustic energy when a diameter of the body lumen (e.g., a renal artery) is within the specified range of diameters that is at least 4 mm, or at least 5 mm. An example of the specified range of diameters is from about 3.0 mm to about 8.0 mm, but is not limited thereto.

In certain embodiments, the specified range of diameters, that is at least 4 mm, or at least 5 mm, includes a lower end of the specified range of diameters and an upper end of the specified range of diameters. In certain such embodiments, the lower end of the specified range of diameters comprises one of about 2.0 mm, about 2.5 mm, or about 3.0 mm, and the upper end of the specified range of diameters comprises one of about 7.5 mm, about 8.0 mm, or about 8.5 mm. Accordingly, the specified range of diameters can be, e.g., from about 2.0 mm to about 7.5 mm, from about 2.0 mm to about 8.0 mm, from about 2.0 mm to about 8.5 mm, from about 2.5 mm to about 7.5 mm, from about 2.5 mm to about 8.0 mm, from about 2.5 mm to about 8.5 mm, from about 3.0 mm to about 7.5 mm, from about 3.0 mm to about 8.0 mm, or from about 3.0 mm to about 8.5 mm. Other variations are also possible and within the scope of embodiments described herein. The term “about,” when used herein to specify a value, means the value+/−10 percent of the value, e.g., “about 3 mm” means 3 mm+/−0.3 mm, and “about 8 mm” means 8 mm+/−0.8 mm.

The amount of acoustic energy emitted by the ultrasound transducer 214 that enters target tissue surrounding the body lumen in which the ultrasound transducer 214 is located is equal to an Acoustic Entry Power multiplied by a duration (T) that the acoustic signal is emitted. The Acoustic Entry Power is based on (aka is dependent on) various factors, including an output power level setting of the excitation source (e.g., 1118), a power efficiency of the system (including the components thereof), a frequency of the acoustic signal emitted by the ultrasound transducer 214, a duration (T) of the acoustic signal emitted by the ultrasound transducer, and an amount of attenuation caused by a medium that is between the ultrasound transducer and the body lumen wall. Where the ultrasound transducer 214 is located within a balloon 108 through which a cooling fluid (e.g., water, sterile water, saline, or D5W) is circulated, the cooling fluid and the balloon material (and potentially, any folds in the balloon material) are the medium between the ultrasound transducer 214 and the body lumen wall. Where the catheter is balloonless (i.e., devoid of a balloon), then blood traveling through the body lumen is the medium between the ultrasound transducer and the body lumen wall. The catheter can include a centering mechanism configured to generally center the ultrasound transducer within the body lumen. In certain embodiments, the centering mechanism is provided by a compliant balloon 108. Alternatively, or additionally, the centering mechanism can comprise one or more flexible baskets attached to a catheter shaft (e.g., 212), or other structures, such as the spiral springs, but is not limited thereto.

In other words, the total energy absorbed (E_{eff}) in the targeted patient tissue (aka the target tissue, the target zone, or the targeted region) surrounding a body lumen (within which the ultrasound transducer 214 is positioned) is the product of an Entry Acoustic Energy (E₀) multiplied by a portion (β) (e.g., percent) of the energy used for ablation in the targeted region, wherein the portion (β) (e.g., percent) of energy used for ablation in the targeted region is dependent on the extent of attenuation caused by the medium between the ultrasound transducer and the body lumen wall. More specifically, E_{eff}=β·E₀=(1−e^{-2αfd})P₀T, where α is the attenuation coefficient (neper/MHz/cm), β the portion (e.g., percent) of energy used for ablation in the targeted region, f is the acoustic frequency, and d is the desired outer lesion boundary (aka lesion depth). The total energy absorbed (E_{eff}) in the targeted region can also be referred to herein as the effective energy (E_{eff}).

The Acoustic Entry Energy (E₀) is the total acoustic power delivered into patient tissue, e.g., through a balloon wall. As the acoustic waves propagate through patient tissue, acoustic power is attenuated and converted into heat, which results in a temperature increase in the tissue. Only a portion (β) (e.g., percent) of the Acoustic Entry Energy (E₀) is absorbed by the targeted region, while the residual portion travels further and is absorbed by untargeted patient tissue beyond the targeted region. As a reminder, energy (such as Acoustic Entry Energy, E₀) is the product of power (such as Acoustic Entry Power, P₀) multiplied by time (aka duration). Thus, the effective energy (E_{eff}), which is the portion of acoustic energy absorbed by the targeted region, is equal to the product of β multiplied by the Acoustic Entry Energy (E₀), i.e., E_{eff}=β·E₀, as was noted above. The value of β can depend on various different parameters, such as, but not limited to, an acoustic frequency and a desired lesion depth d.

To keep (aka maintain) the same lesion boundary d (aka lesion depth), E_{eff} should be kept constant. This statement is true when the treatment time (aka duration) T does not change significantly, and when an impact from heat conduction does not change significantly. It is noted that more total energy or effective energy is generally required if the treatment time (aka duration) T is increased significantly to compensate for the heat loss due to conduction. Table 3 below shows the Acoustic Entry Power for various different ultrasound frequencies, assuming a desired lesion depth (d) of 4 mm, and a treatment duration (T) of 7 seconds.

TABLE 3

| d = 4 mm, T = 7 seconds | | | | |
|---|---|---|---|---|
| α = 0.058 neper/Mhz/cm | 7 MHz | 9 MHz | 12 MHz | 15 MHz |
| β | 27.73% | 34.14% | 42.70% | 50.14% |
| Acoustic Entry Power (P₀) | 32.0 W | 26.0 W | 20.8 W | 17.7 W |
| | | (reference value | | |
| | | for others) | | |

Table 4 below shows the Acoustic Entry Power for various different ultrasound frequencies, assuming a desired lesion depth (d) of 6 mm, and a treatment duration (T) of 7 seconds. As can be appreciated from a comparison between Table 4 and Table 3, a higher Acoustic Entry Power of 35.6 W should be used when there is a desire to produce a lesion depth of 6 mm, compared to an Acoustic Entry Power of 26.0 W that may be used where the desired lesion depth is 4 mm (as may be appropriate more distal, i.e., closer to the kidneys).

TABLE 4

| d = 6 mm, T = 7 seconds | | | | |
|---|---|---|---|---|
| α = 0.058 neper/Mhz/cm | 7 MHz | 9 MHz | 12 MHz | 15 MHz |
| ß | 38.57% | 46.55% | 56.62% | 64.80% |
| Acoustic Entry Power (E₀) | 41.8 W | 34.6 W | 28.4 W | 24.9 W |
| | | (reference | | |
| | | value for | | |
| | | others) | | |

Table 5 below shows the Acoustic Entry Power for various different ultrasound frequencies, assuming a desired lesion depth (d) of 6 mm, and a treatment duration (T) of 10 seconds. As can be appreciated from a comparison between Table 5 and Table 4, a lower Entry Power of 24.2 W should be used when there is a desire to produce a lesion depth of 6 mm where the duration that the Acoustic Entry Power is delivered is 10 seconds, compared an Acoustic Entry Power of 36.4 W that may be delivered for a shorter duration of 7 seconds to produce the same desired lesion depth of 6 mm.

TABLE 5

| d = 6 mm, T = 10 seconds | | | | |
|---|---|---|---|---|
| α = 0.058 neper/Mhz/cm | 7 MHz | 9 MHz | 12 MHz | 15 MHz |
| ß | 38.57% | 46.55% | 56.62% | 64.80% |
| Acoustic Entry Power (E₀) | 29.2 W | 34.6 W * 7/10 = | 19.9 W | 17.4 W |
| | | 24.2 W | | |
| | | (reference value | | |
| | | for others) | | |

In certain single power embodiments, the controller 104 is configured to control the excitation source to cause the ultrasound transducer 214 to emit the same amount of acoustic energy (when the diameter of the segment of the body lumen, within which the ultrasound transducer is located, is within the specified range of diameters) by controlling the excitation source 1118 so that the output power level setting is about the same, the frequency of the acoustic energy emitted by the transducer 214 is about the same (e.g., about 9 MHZ), and the duration of the acoustic energy emitted by the transducer 214 is about the same (e.g., about 7 seconds). In certain such embodiments, a frequency of the acoustic energy is about 9 MHz, the duration of the acoustic power delivery is about 7 seconds, and the Acoustic Entry Power is about 34.6 W. In certain single power embodiments, the catheter 102 includes a balloon 108 in which is located the ultrasound transducer 214, wherein the balloon 108 may be a compliant balloon, example details of which were described above with reference to FIGS. 5-9. In other single power embodiments the catheter is balloonless.

For Tables 3, 4 and 5, the values shown therein are example values for a tissue treatment system 100 where the ultrasound transducer 214 is located within a balloon 108 through which a cooling fluid is circulated. As noted above, where the catheter is balloonless (i.e., devoid of a balloon), blood traveling through the body lumen is the medium between the ultrasound transducer and the body lumen wall. By contrast, when the catheter includes a balloon 108 within which is located the ultrasound transducer 214, the medium between the ultrasound transducer 214 and the body lumen wall is the cooling fluid (being circulated through the balloon 108) and the balloon material (from which the balloon 108 is made). The attenuation coefficient of blood is greater (e.g., at least 10× greater) than the attenuation coefficient of a typical cooling fluid, and thus, the values shown in the Tables 3, 4 and 5 would differ for balloonless embodiments.

Where the catheter 102 includes a balloon 108 within which is located the ultrasound transducer 214, the tissue treatment system 100 can also include a fluid supply subsystem 1130 that is configured to circulate cooling fluid through the balloon 108, and the controller 104 can be configured to control the fluid supply subsystem 1130. In such a system, the amount of Acoustic Entry Power is also based on a flowrate of the cooling fluid circulated through the balloon 108. In certain single power embodiments, the controller 104 can be configured to control the fluid supply subsystem 1130 so that the flowrate of the cooling fluid circulated through the balloon is the same when the diameter of the body lumen is within the specified range of diameters (e.g., from about 3 mm to about 8 mm). The flowrate of the cooling fluid circulated through the balloon 108 can be, e.g., within a flowrate range of about 5 mL/min to about 40 mL/min, and in a specific embodiment is in a flowrate range of about 10 mL/min to about 15 mL/min. Unless stated otherwise, it is assumed that a temperature of the cooling fluid is a room temperature of the room where the tissue treatment system 100 is located, however it is also possible that the temperature of the cooling fluid can be modified if so desired, e.g., using a cooling element, or the like.

It has been found advantageous to configure the controller 104 to control the fluid supply system 1130 to circulate the fluid through the balloon 108 a predetermined amount of time after at least the first amount of energy has been emitted by the ultrasound transducer 214. When the energy emission is stopped, the fluid supply system 1130 may thus continue to operate for the predetermined amount of time so as to ensure an efficient cooling and avoid undesired clinical outcomes (e.g., in terms of lesion depths). The predetermined amount of time may be within a range of 0.5 secs to 20 secs, such as 2 secs to 12 secs, in particular 5 secs to 9 secs. While such a “post-emission cooling” is particularly useful for system operation in the lower subrange, the controller 104 could also be configured to control the fluid supply system 1130 to circulate the fluid through the balloon 108 a predetermined amount of time after at least the second amount of energy has been emitted by the ultrasound transducer 214 (i.e., upon system operation in the upper subrange). When the energy emission for the upper subrange is stopped, the fluid supply system 1130 may thus continue to operate for the predetermined amount of time. The predetermined amount of time is within a range of 0.5 secs to 20 secs, such as 2 secs to 12 secs, in particular 5 secs to 9 secs. Such a “post-emission cooling” can also be applied in the context of the single-power strategy.

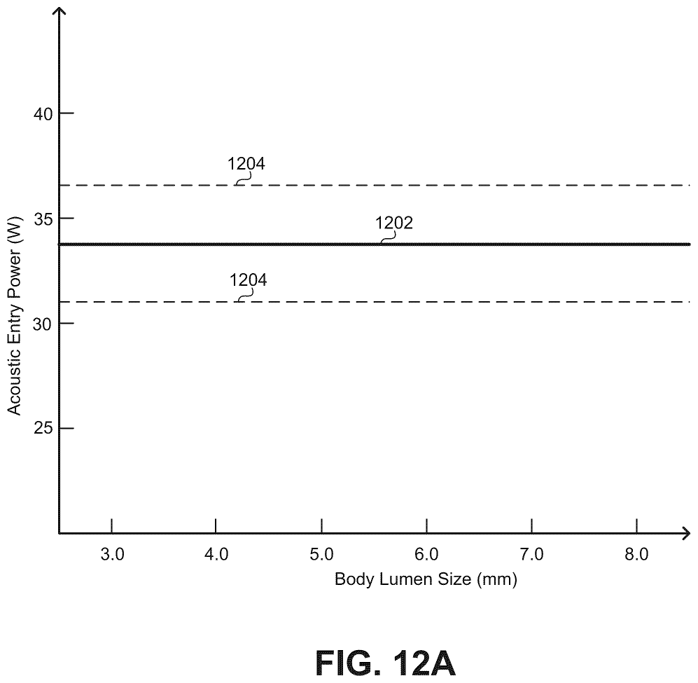

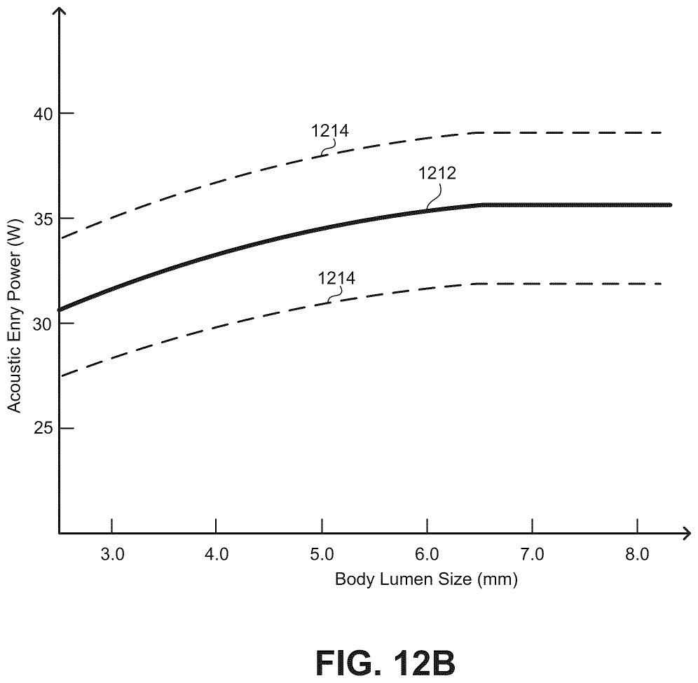

FIG. 12A is a graph of Acoustic Entry Power in watts (W) versus body lumen size in millimeters (mm), corresponding to an example implementation of the single power embodiments. The straight line 1202 in FIG. 12A illustrates the Acoustic Entry Power (W) remaining about the same over the range of body lumen diameters from about 3.0 mm to about 8.0 mm, and the dashed lines 1204 corresponds to a variation of +/−10%. So long as the Acoustic Entry Power remains within the dashed lines 1204 (e.g., in view of uncertainties such as tolerances) it can be said that the Acoustic Entry Power remains about the same, and more specifically, remains about 34.6 W.

FIG. 12B is a graph of Acoustic Entry Power in watts (W) versus body lumen size in millimeters (mm) corresponding to another example implementation of the single power embodiments. The curved line 1212 in FIG. 12B illustrates how the acoustic power may be somewhat lower for smaller body lumen diameters than for larger body lumen diameters over the range of body lumen diameters from about 3.0 mm to about 8.0 mm, and the dashed lines 1214 corresponds to a variation of +/−10%. For the graph of FIG. 12B it is assumed that the ultrasound transducer 214 is located within a compliant balloon 108, e.g., a balloon comprising Pellethane having a Shore D durometer of 55 and having a nominal balloon diameter (e.g., of 6.5 mm) and a corresponding nominal balloon wall thickness. In accordance with certain embodiments, a compliant balloon disclosed in U.S. patent application Ser. No. 17/812,884, filed Jul. 15, 2022, titled CATHETER HAVING COMPLIANT BALLOON, published as US20230026169, which is incorporated herein by reference, may also be used. In accordance with certain embodiments, a balloon, such as a balloon disclosed in U.S. patent application Ser. No. 17/812,884 that includes several helical folds extending about the ultrasound transducer between the proximal balloon end and the distal balloon end, and being held in place by the torsion member, such as disclosed in U.S. Provisional Patent Application No. 63/482,463, titled TISSUE TREATMENT CATHETER HAVING TORSION MEMBER, filed Jan. 31, 2023 may be used.

In FIG. 12B, the acoustic power may be lower for smaller body lumen diameters due to one or more folds in the compliant balloon 108, which one or more folds is/are present when the compliant balloon 108 is partially inflated such that its diameter is less than a nominal balloon diameter (e.g., of 6.5 mm) of the compliant balloon 108, at least partially attenuating some of the acoustic power emitted by the ultrasound transducer 214. This can reduce an amount of the acoustic power that passes through balloon 108 when a diameter of the body lumen in which the compliant balloon 108 is in apposition is within a smaller diameter subset (e.g., from about 3.0 mm to about 6.5 mm) of the specified range of diameters (e.g., from about 3.0 mm to about 8.0 mm), compared to when the compliant balloon 108 is inflated such that its diameter is at least the nominal balloon diameter (e.g., of 6.5 mm) of the compliant balloon 108 and the diameter of the body lumen in which the compliant balloon 108 is in apposition is within a larger diameter subset (e.g., from about 6.5 mm to about 8 mm) of the specified range of diameters (e.g., from about 3.0 mm to about 8.0 mm). More specifically, the aforementioned folds (e.g., wrinkles) in the compliant balloon 108 can increase reflections and/or scattering of ultrasound signals emitted by the ultrasound transducer 214, which increases a propagation length that the ultrasound signals travel before exiting the balloon 108 and entering target tissue surrounding the body lumen. Additionally, the folds effectively increase the balloon thickness where folds (e.g., wrinkles) reside. Because the thickness of the balloon material affects how much attenuation is caused by the balloon (i.e., the greater the thickness of the balloon material the greater the attenuation caused), the effective increase in the balloon thickness caused by the folds also contributes to the acoustic power attenuation. Both of these factors cause a higher percentage of acoustic power loss before the acoustic power propagates into the target tissue compared to using a balloon of a same size without folds (e.g., without wrinkles). Examples of the folds (aka wrinkles) in the compliant balloon 108 are shown in and described above with reference to FIGS. 8A-8F. Certain embodiments take advantage of the folds to help deliver appropriate amounts of acoustic power to tissue being treated.

More generally, the folds (e.g., 800, 808, and/822, but not limited thereto) in the compliant balloon 808, which are present when the compliant balloon is partially inflated such that its diameter is less than the nominal balloon diameter of the compliant balloon, are configured to attenuate more of the acoustic power emitted by the ultrasound transducer, and thereby are configured to allow less of the acoustic power to pass through the compliant balloon 108 when the compliant balloon is in apposition with a body lumen segment having a diameter that is within a lower diameter subset (e.g., below 5 mm) of the specified range of diameters, compared to when the compliant balloon is in apposition with a body lumen segment that is within a larger diameter subset (e.g., equal to or greater than 5 mm) of the specified range of diameters.

In certain embodiments, the compliant balloon 108 stretches when the compliant balloon 108 is inflated beyond the nominal balloon diameter (e.g., of about 6.5 mm), which causes a balloon wall thickness to get thinner than the nominal balloon wall thickness. This can result in less attenuation of the acoustic power emitted by the ultrasound transducer 214 when a diameter of the body lumen in which the compliant balloon 108 is in apposition with a body lumen whose diameter is within a larger diameter subset of the specified range of diameters, compared to when the diameter of the body lumen in which the compliant balloon 108 is in apposition is within a lower diameter subset of the specified range of diameters. In other words, another reason that the Acoustic Entry Power may be greater for larger body lumen sizes, compared to smaller body lumen sizes, is that a balloon wall thickness may be reduced the more the balloon is inflated, with the reduced wall thickness causing less attenuation to acoustic power.

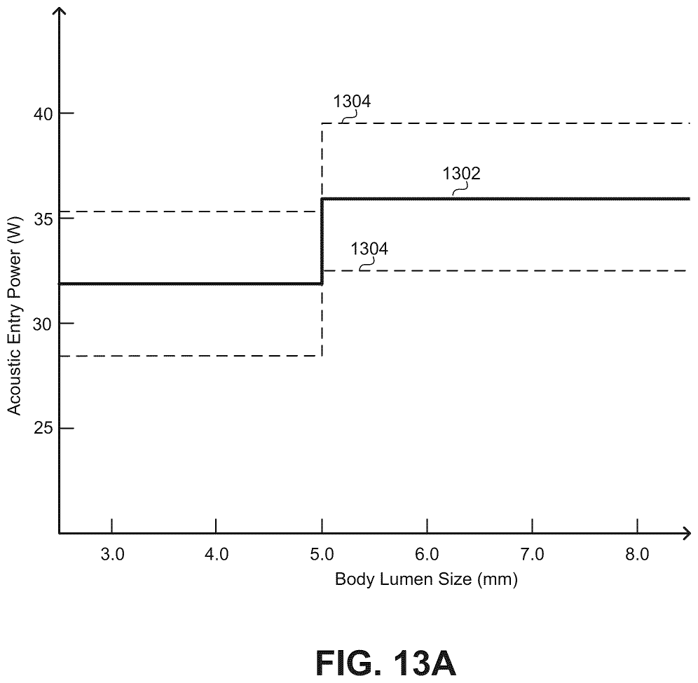

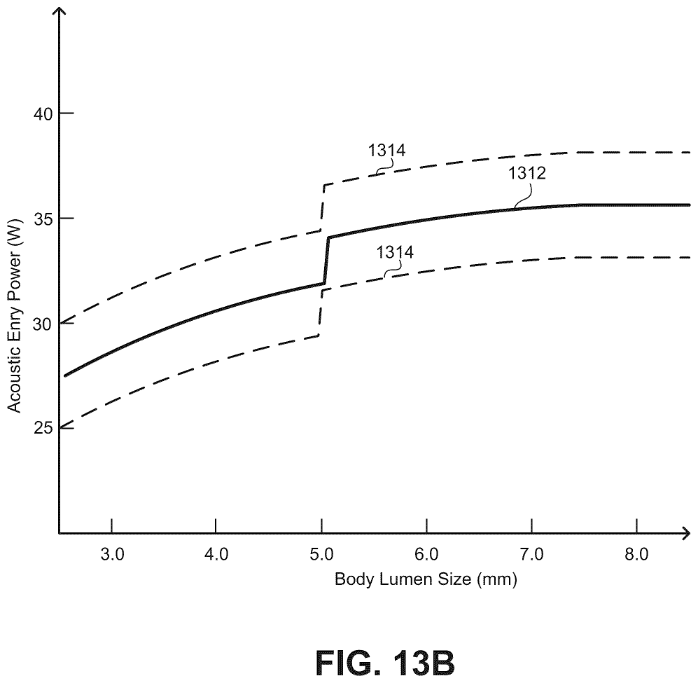

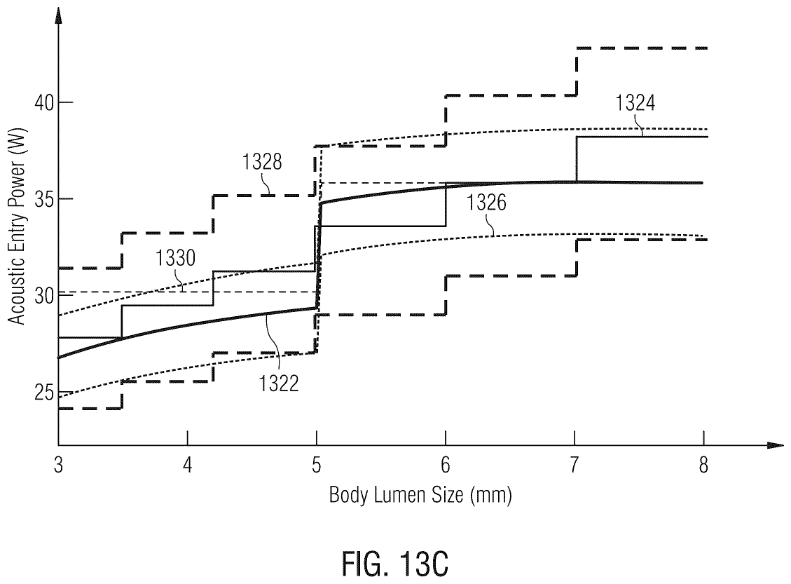

FIG. 13A is a graph of Acoustic Entry Power in watts (W) versus body lumen size in millimeters (mm), corresponding to an example implementation of the two power embodiments. The stepped line 1302 in FIG. 13A illustrates the Acoustic Entry Power (W) being at about a first magnitude (e.g., about 32.0 W) when the diameter of the body lumen is within the lower subrange of diameters from about 3.0 mm to about 4.9 mm, and Acoustic Entry Power (W) being at about a second magnitude (e.g., about 35.8 W) when the diameter of the body lumen is within the upper subrange of diameters from about 5.0 mm to about 8.0 mm, and the dashed lines 1304 corresponds to a variation of +/−10% (e.g., in view of uncertainties such as tolerances).

FIG. 13B is a graph of Acoustic Entry Power in watts (W) versus body lumen size in millimeters (mm) corresponding to another example implementation of the two power embodiments. The curved line 1312 in FIG. 13B illustrates how the acoustic power may be somewhat lower for smaller body lumen diameters than larger lumen diameters within the lower subrange of diameters from about 3.0 mm to about 4.9 mm, and may be somewhat lower for smaller body lumen diameters than larger lumen diameters within the upper subrange of diameters from about 5.0 mm to about 8.0 mm. The dashed lines 1314 corresponds to a variation of +/−10%. For the graph of FIG. 13B it is assumed that the ultrasound transducer 214 is located within a compliant balloon 108 having nominal balloon diameter (e.g., of 6.75 mm) and a corresponding nominal balloon wall thickness.

For similar reasons to those described above with reference to FIG. 12B, the acoustic power may be lower for smaller body lumen diameters than larger lumen diameters within the lower subrange of diameters from about 3.0 mm to about 4.9 mm due to one or more folds in the compliant balloon 108, which is/are present when the compliant balloon 108 is partially inflated such that its diameter is less than a nominal balloon diameter (e.g., of 6.5 mm) of the compliant balloon 108, at least partially attenuating some of the first amount of acoustic power emitted by the ultrasound transducer 214. Similarly, the acoustic power may be lower for smaller body lumen diameters than larger lumen diameters within the upper subrange of diameters from about 5.0 mm to about 8.0 mm due to one or more folds in the compliant balloon 108, which one or more folds is/are present when the compliant balloon 108 is partially inflated such that its diameter is less than a nominal balloon diameter (e.g., of 6.5 mm) of the compliant balloon 108, at least partially attenuating some of the second acoustic power emitted by the ultrasound transducer 214. This is because, as explained above, the folds can increase reflections and/or scattering of ultrasound signals emitted by the ultrasound transducer 214, which increases a propagation length that the ultrasound signals travel before exiting the balloon 108 and entering target tissue surrounding the body lumen. Additionally, the folds effectively increase the balloon thickness where folds (e.g., wrinkles) reside. As noted above, another reason that the Acoustic Entry Power may be greater for larger body lumen sizes, compared to smaller body lumen sizes, is that a balloon wall thickness may be reduced the more the balloon is inflated past its nominal inflation diameter, with the reduced wall thickness causing less attenuation to acoustic power.

FIG. 13C is a graph of Acoustic Entry Power in watts (W) versus body lumen size in millimeters (mm) corresponding to another example implementation of the two power embodiments. The thicker solid curved line 1322 in FIG. 13C illustrates how the acoustic power may be somewhat lower for smaller body lumen diameters than larger lumen diameters within the lower subrange of diameters from about 3.0 mm to about 4.9 mm, and may be somewhat lower for smaller body lumen diameters than larger lumen diameters within the upper subrange of diameters from about 5.0 mm to about 8.0 mm. For the graph of FIG. 13C it is assumed that the ultrasound transducer 214 is located within a compliant balloon 108 having nominal balloon diameter (e.g., of 6.5 mm) and a corresponding nominal balloon wall thickness.

For similar reasons to those described above with reference to FIGS. 12B and 13B, the acoustic power (e.g., in terms of patient entry power at a constant nominal power setting by the controller 104) may be lower for smaller body lumen diameters than larger lumen diameters within the lower subrange of diameters from about 3.0 mm to about 4.9 mm due to one or more folds in the compliant balloon 108, which is/are present when the compliant balloon 108 is partially inflated such that its diameter is less than a nominal balloon diameter (e.g., of 6.5 mm) of the compliant balloon 108, at least partially attenuating some of the first amount of acoustic power emitted by the ultrasound transducer 214. Similarly, the acoustic power (e.g., in terms of patient entry power at a constant nominal power setting by the controller 104) may be lower for smaller body lumen diameters than larger lumen diameters within the upper subrange of diameters from about 5.0 mm to about 8.0 mm due to one or more folds in the compliant balloon 108, which one or more folds is/are present when the compliant balloon 108 is partially inflated such that its diameter is less than a nominal balloon diameter (e.g., of 6.5 mm) of the compliant balloon 108, at least partially attenuating some of the second acoustic power emitted by the ultrasound transducer 214. The dashed line 1330 illustrates for comparison purposes the power curve (e.g., in terms of the nominal power setting by the controller 104) when the attenuation caused by the “folds effect” described above (and possibly further attenuating effects) is not considered. As becomes apparent from a comparison of lines 1322 and 1330, the “folds effect” is more significant for body lumens sizes (and inflated balloon diameters) below the nominal balloon diameter (here: of 6.5 mm), since when the nominal balloon diameter is reached upon inflation of the balloon 108, the folds (e.g., in terms of wrinkles 800) disappear. In practice, it has been found to be advantageous if the nominal balloon diameter is selected greater than the specified intermediate diameter (here: 5 mm) that separates the lower subrange from the upper subrange.

FIG. 13C also shows a comparison of the two-power strategy discussed above and a more complex six-power strategy using six different catheters. In FIG. 13C, the thin solid line 1324 illustrates the acoustic power (e.g., in terms of acoustic patient power) respectively output by each of the six the catheters designed specifically for six particular body lumen sizes and having respective (non-compliant) balloon diameters of 3.5 mm, 4.2 mm, 5.0 mm, 6.0 mm, 7.0 mm, and 8.0 mm inflated to their full sizes. The dotted line 1326 and the dashed line 1328 illustrate the power uncertainties in the acoustic power output for the two-power strategy (line 1322) and the six-power strategy (line 1324), respectively. Those uncertainties are due to inherent system tolerances (e.g., of the excitation source) and other effects. As becomes apparent from the lines 1326, 1328, it has been found that by implementing a slightly tighter tolerance control for the two-power strategy, the resulting (smaller) uncertainties of the two-power strategy can be kept within the power envelope of the six-power strategy, so that consistent clinical outcomes (e.g., in terms of safety and effectiveness) can be ensured.

As further becomes apparent from the lines 1326, 1328 indicative of the uncertainties of the two-power strategy and the six-power strategy, respectively, the attenuation by the “folds effect” is indeed helpful especially for smaller lumen sizes in the lower and upper subranges to make sure that the acoustic entry power (with its inherent uncertainties) of the two-power strategy does not exceed the power envelope of the (well tested) six-power strategy. If the “folds effect” was not present in the two-power strategy (see line 1330 which illustrates the two-power approach without attenuation by folds), the acoustic power (with its inherent uncertainty, not illustrated in FIG. 13C) would be too high especially in the lower vessel size regions of the lower subrange and the upper subrange (and when assuming that the remaining parameters such as frequency and duration of energy emission remain constant). These findings prove that the two-power strategy indeed is a practical solution (e.g., because consistent lesion depths and, thus, consistent clinical outcomes can be ensured).

The overall power uncertainty, or tolerance, is defined by two major factors: the power tolerance of the excitation source (also called “generator”) and the uncertainty caused by the catheter acoustic efficiency. Also the power measurement accuracy may need to be considered. The power tolerance of the excitation source was found to be the largest contributor to the overall power uncertainty. At a low power setting of 10 watts of a conventional excitation source, the power tolerance can be in the range of +/−25% and will decrease with higher power settings. At power settings as generally proposed herein of about 25 to 35 watts, the power tolerance will be in the range of +/−13%. As such, for the two-power strategy, a power tolerance below +/−12% and in particular below +/−10% or below +/−8% can be implemented. Such a tighter tolerance control becomes possible by, for example, using higher quality components (e.g., in terms of the excitation source) and power control loops. Taking into account the total power uncertainty (including, for example, uncertainty caused by the catheter acoustic efficiency and possibly power measurement uncertainties), a power tolerance below+/−26% and in particular below+/−21% or below+/−16% can be implemented.

In view of the desired “folds effect”, it can be advantageous not to reduce the tolerances below a certain threshold so that power fluctuations caused by a certain randomness of how and where the folds are arranged and unfold can still be accommodated. Despite an apparent randomness resulting in regard to the folds (e.g., when taking the form of wrinkles), it has been found that there is a predictable, generally linear relationship between the acoustic power attenuation by the folds on the on hand and a “degree of foldedness” on the other hand, with the “foldedness” being mathematically defined as ((nominal balloon diameter/inflated balloon diameter−1)*100%). When the inflated balloon diameter is greater than the nominal balloon diameter, the “degree of foldedness” is defined to be zero. The acoustic power attenuation can be defined as (1-measured acoustic power/expected acoustic power without folds)*100%. The expected acoustic power was derived from the mean value of repeated measurements using balloons without folds, whereas the measured acoustic power was measured for similar balloons with folds. It has additionally been found that any remaining randomness in the power distribution caused by the folds is compensated by heat conduction in the tissue. These outcomes provide a consistent physical foundation for the two-power strategy.

As also becomes apparent from FIG. 13C, in particular the combined contributions of a tight power control and the “folds effect” pave the way for the two-power strategy. The lines 1326, 1328 indicative of the uncertainties of the two-power strategy and the six-power strategy, respectively, further illustrate that it is indeed beneficial to select the nominal balloon diameter (here: 6.5 mm) greater than the specified intermediate diameter (here: 5 mm) that separates the lower subrange from the upper subrange. Such a selection helps to ensure that the tolerance regime of the two-power strategy remain within the power uncertainty envelope of the six-power strategy.

In the two-power strategy it is advantageous to select the first amount of acoustic energy based on an acoustic entry power within a first range from about 25.0, 27.5 or 30.0 to about 33.0 watts and to select the second amount of acoustic energy based on an acoustic entry power within a second range from about 32.0 or 33.1 to about 39.0 watts. The duration of the power delivery in the lower subrange can be selected such that the first amount of acoustic energy lies within a range between 140J to 240J, or between 165J and 215J (e.g., around 190J). The duration of the power delivery in the higher subrange can be selected such that the second amount of acoustic energy lies within a range between 180J to 270J, or between 200j and 250J (e.g., around 225J). In these examples, the generator frequency can be set to around 9 MHz. The energy amounts can be reduced by ca. 15 to 25% in case of a higher frequency of around 12 MHz and increased by ca. 20 to 35% for lower frequencies of around 6 MHz. In all the variants discussed herein, energy measurements can be performed in accordance with IEC 61161 Edition 3.0, 2013-01 or BS EN 62555:2014.

In accordance with certain embodiments, the controller 104 is configured to automatically determine an estimate of the diameter of the body lumen, e.g., using one of the techniques described in commonly assigned U.S. patent application Ser. No. 17/812,973, titled METHODS AND SYSTEMS FOR DETERMINING BODY LUMEN SIZE, filed Jul. 15, 2022, published as US20230026504, which is incorporated herein by reference. Additionally, the controller 104 is configured to control the excitation source 1118 to cause the ultrasound transducer 214 to emit the first amount of acoustic energy based on a determination that the estimate of the diameter of the body lumen is within the lower subrange of the specified range of diameters, and control the excitation source 1118 to cause the ultrasound transducer 214 to emit the second amount of acoustic energy based on a determination that the estimate of the diameter of the body lumen is within the upper subrange of the specified range of diameters.

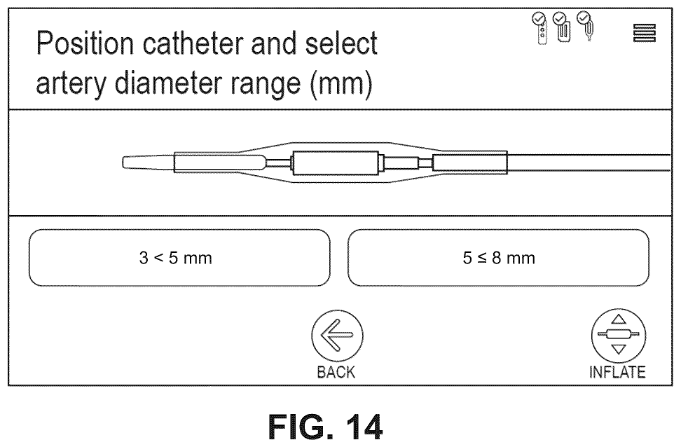

In accordance with certain embodiments, the user interface 1116 of the system 100 allows a user to specify whether the diameter of the body lumen is within the lower subrange of the specified range of diameters or within the upper subrange of the specified range of diameters. An example of such a user interface 1116 is shown in FIG. 14. In an embodiment, the user interface 1116 is further configured to display a warning message when an automatically determined estimate of the diameter of the body lumen is not within the subrange input by the user. It is also possible that the user interface 1116 is configured to allow a user to specify the type of body lumen. For example, the controller can store in the memory 1114 a plurality of different types of body lumens and whether each type of body lumen is within the lower subrange of the specified range of diameters or the upper subrange of the specified range of diameters. The plurality of different types of body lumens can include, e.g., a main renal artery, an accessory renal artery, and a renal artery branch, but is not limited thereto. In certain such embodiments, the controller 104 is further configured to control the excitation source 1118 to cause the ultrasound transducer 214 to emit the first amount of acoustic energy when the diameter of the body lumen is specified, using the user interface, to be within the lower subrange of the specified range of diameters, and control the excitation source 1118 to cause the ultrasound transducer 214 to emit the second amount of acoustic energy when the diameter of the body lumen is specified, using the user interface 1116, to be within the upper subrange of the specified range of diameters. The user interface 1116 can also be configured to display a warning message when an automatically determined estimate of the diameter of the body lumen is not within the subrange input by the user.

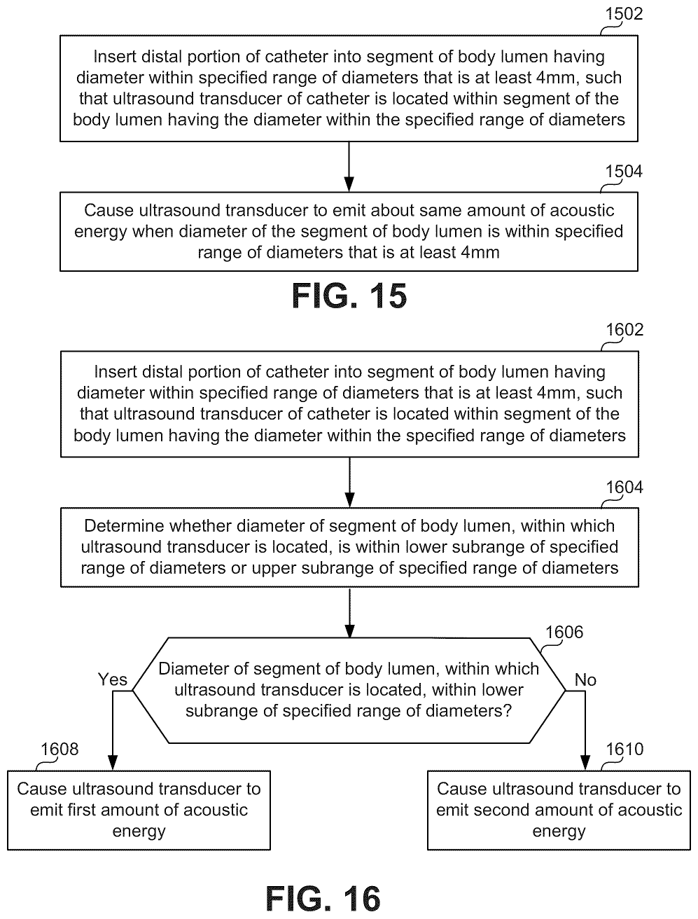

FIG. 15 is a high level flow diagram used to summarize a single power method for use with a tissue treatment system having a catheter 102 that includes a distal portion (e.g., a shaft 212) on which is located an ultrasound transducer 214. Referring to FIG. 15, step 1502 involves inserting the distal portion of the catheter 102 into a segment of body lumen having a diameter within a specified range of diameters that is at least 4 mm, such that the ultrasound transducer is located within the segment of the body lumen having the diameter within the specified range of diameters that is at least 4 mm. For example, the range of diameters can be from about 3.0 mm to about 8.0 mm, but is not limited thereto. Further details of such ranges were described above, and thus, need not be repeated. Step 1504 involves causing the ultrasound transducer 214 to emit about a same amount of acoustic energy when the diameter of the segment of the body lumen is within the specified range of diameters that is at least 5 mm, e.g., from about 3.0 mm to about 8.0 mm. Example amounts of acoustic energy that could be used in the single power embodiments were described above, and thus, need not be repeated.

As was explained above, the distal portion of the catheter 102 can include a compliant balloon 108 within which is located the ultrasound transducer 214, wherein the compliant balloon 108 is configured such that when the compliant balloon 108 is partially inflated, such that its diameter is less than a nominal balloon diameter of the compliant balloon, the compliant balloon includes one or more folds, examples of which were described above. In such an embodiment, the method can also include causing inflation of the compliant balloon 108 so that the compliant balloon is in apposition with a body lumen wall of the segment of the body lumen within which is located the ultrasound transducer 214. The method can also include utilizing the one or more folds in the compliant balloon to at least partially attenuate some of the acoustic power emitted by the ultrasound transducer 214, and thereby reduce an amount of the acoustic power that passes through the compliant balloon 108 when the diameter of the segment of the body lumen in which the compliant balloon is in apposition is within a smaller diameter subset of the specified range of diameters, compared to when the compliant balloon is inflated such that its diameter is at least the nominal balloon diameter of the compliant balloon and the diameter of the segment of the body lumen in which the compliant balloon is in apposition with is within a larger diameter subset of the specified range of diameters.

FIG. 16 is a high level flow diagram used to summarize of a two power method for use with a tissue treatment system having a catheter 102 that includes a distal portion (e.g., a shaft 212) on which is located an ultrasound transducer 214. Referring to FIG. 16, step 1602 involves inserting the distal portion of the catheter 102 into a segment of body lumen having a diameter within a specified range of diameters that is at least 4 mm, such that the ultrasound transducer is located within the segment of the body lumen having the diameter within the specified range of diameters that is at least 4 mm. For example, the range of diameters can be from about 3.0 mm to about 8.0 mm, but is not limited thereto. Further details of such ranges were described above, and thus, need not be repeated. Step 1604 involves determining whether the diameter of the segment of the body lumen, within which the ultrasound transducer 214 is located, is within a lower subrange of the specified range of diameters or an upper subrange of the specified range of diameters. For example, the lower subrange can be from about 3.0 mm to about 4.9 mm, and the upper subrange can be from about 5.0 mm to about 8.0 mm, but are not limited thereto. For another example, the lower subrange can be from about 3.0 mm to about 4.5 mm, and the upper subrange can be from about 4.6 mm to about 8.0 mm, but are not limited thereto. Further details of such ranges and subranges were described above, and thus, need not be repeated. There is a determination of step 1606, which results in flow going to either step 1608 or 1610. Step 1608 involves causing the ultrasound transducer to emit a first amount of acoustic energy when the diameter of the segment of the body lumen within which the ultrasound transducer 214 is located is determined to be within the lower subrange (e.g., from about 3.0 mm to about 4.9 mm) of the specified range of diameters. By contrast, step 610 involves causing the ultrasound transducer to emit a second amount of acoustic energy, which is greater than the first amount of acoustic energy, when the diameter of the segment of the body lumen within which the ultrasound transducer is located is determined to be within the upper subrange (e.g., from about 5.mm to about 8.0 mm) of the specified range of diameters.

In accordance with certain embodiments, the determining at step 1604 (of whether the diameter of the segment of the body lumen, within which the ultrasound transducer is located, is within the lower subrange of the specified range of diameters or the upper subrange of the specified range of diameters) is determined by a user, e.g., using fluoroscopy of some other visualization technique, or the like, and entered by the user into the tissue treatment system using a user interface, e.g., 1116, of the tissue treatment system. An example of such a user interface is shown in FIG. 14, which was discussed above.

In accordance with certain embodiments, the determining at step 1604 (of whether the diameter of the segment of the body lumen, within which the ultrasound transducer is located, is within the lower subrange of the specified range of diameters or the upper subrange of the specified range of diameters) is determined by accepting an indication, from a user via a user interface, e.g., 1116, of which one of a plurality of different types of body lumens the ultrasound transducer is located. Whether the diameter of the segment of the body lumen (within which the ultrasound transducer is located) is within the lower subrange of the specified range of diameters or the upper subrange of the specified range of diameters, is then determined based on the indication accepted via the user interface. The plurality of different types of body lumens can include, for example, a main renal artery, an accessory renal artery, and a renal artery branch, but are not limited thereto.

In accordance with certain embodiments, the determining at step 1604 (of whether the diameter of the segment of the body lumen, within which the ultrasound transducer is located, is within the lower subrange of the specified range of diameters or the upper subrange of the specified range of diameters) is automatically determined by the tissue treatment system, e.g., using one of the techniques described in U.S. patent application Ser. No. 17/812,973, titled METHODS AND SYSTEMS FOR DETERMINING BODY LUMEN SIZE, filed Jul. 15, 2022, published as US20230026504, which is incorporated herein by reference.

As was explained above, the distal portion of the catheter 102 can include a compliant balloon 108 within which is located the ultrasound transducer 214, wherein the compliant balloon 108 is configured such that when the compliant balloon 108 is partially inflated, such that its diameter is less than a nominal balloon diameter of the compliant balloon, the compliant balloon includes one or more folds, examples of which were described above. In such an embodiment, the method can also include causing inflation of the compliant balloon 108 so that the compliant balloon is in apposition with a body lumen wall of the segment of the body lumen within which is located the ultrasound transducer 214. The method can also include utilizing the one or more folds in the compliant balloon to at least partially attenuate some of the acoustic power emitted by the ultrasound transducer, and thereby reduce an amount of the acoustic power that passes through the compliant balloon when the diameter of the segment of the body lumen in which the compliant balloon is in apposition is within a smaller diameter subset of the specified range of diameters, compared to when the compliant balloon is inflated such that its diameter is at least the nominal balloon diameter of the compliant balloon and the diameter of the segment of the body lumen in which the compliant balloon is in apposition with is within a larger diameter subset of the specified range of diameters.

The body lumen, with which the methods described with reference to FIGS. 15 and 16 may be used, can be a renal artery, but is not limited thereto. For example, the body lumen can be one of: a vein; a pulmonary artery; a vascular lumen; a celiac artery; a common hepatic artery; a proper hepatic artery; a gastroduodenal artery; a hepatic artery; a splenic artery; a gastric artery; a blood vessel; a nonvascular lumen; an airway; a sinus; an esophagus; a respiratory lumen; a digestive lumen; a stomach; a duodenum; a jejunum; or a cancer tissue.

In accordance with certain embodiments, the body lumen into which a catheter of a system described above is inserted into, and used to denervate nerves in tissue surrounding the body lumen, is a renal artery and the nerves comprise renal nerves innervating a kidney. Where a denervation procedure described herein is being performed using a catheter (e.g., 102) inserted into a renal artery type of body lumen, the disease being treated using the denervation procedure can be hypertension or some other disorder associated with elevated sympathetic nerve activity, as can be appreciated from the above discussion. However, it is noted that embodiments of the present technology described herein can alternatively be used in the performance of denervation procedures (and/or other tissue treatment procedures) using catheters that are inserted into other types of body lumens, besides a renal artery, to treat other types of diseases besides hypertension. For example, such other types of body lumens include a vein, a pulmonary artery, a vascular lumen, a celiac artery, a common hepatic artery, a proper hepatic artery, a gastroduodenal artery, a hepatic artery, a splenic artery, a gastric artery, a blood vessel, a nonvascular lumen, an airway, a sinus, an esophagus, a respiratory lumen, a digestive lumen, a stomach, a duodenum, a jejunum, a cancer tissue, a tumor, an intestine, and a urological lumen, but are not limited thereto. Examples of other types of diseases that can be treated using an embodiment of the present technology include pulmonary hypertension, diabetes, obesity, nonalcoholic fatty liver disease, heart failure, end-stage renal disease, digestive disease, cancers, tumors, pain, asthma or chronic obstructive pulmonary disease (COPD), but are not limited thereto. As has become apparent from the above description, in particular in the context of the two-power strategy as illustrated in FIG. 13C, a tissue treatment system is presented. The system comprises a catheter including a distal portion on which is located an ultrasound transducer, wherein the catheter is configured such that at least the distal portion of the catheter is insertable into a segment of a body lumen having a diameter within a specified range of diameters, wherein the range of diameters has a lower subrange and an upper subrange. The system further comprises an excitation source configured to selectively provide energy to the ultrasound transducer of the catheter and a controller communicatively coupled to the excitation source, the controller configured to control the excitation source to cause the ultrasound transducer to emit two different amounts of acoustic energy, which include a first amount of acoustic energy and a second amount of acoustic energy, which is greater than the first amount of acoustic energy. The controller is configured to control the excitation source to cause the ultrasound transducer to selectively emit the first amount of acoustic energy or the second amount of acoustic energy upon determining that the diameter of a segment of a body lumen to be treated is within the lower subrange or the upper subrange, respectively.

The controller may be configured to control the excitation source to cause the ultrasound transducer to emit the first amount of acoustic energy upon determining that the diameter of a segment of a body lumen to be treated is within the lower subrange. Similarly, the controller may be configured to control the excitation source to cause the ultrasound transducer to emit the second amount of acoustic energy upon determining that the diameter of a segment of a body lumen to be treated is within the upper subrange. In some variants, the controller is configured to have an operational mode in which only the first amount and the second amount of acoustic energy can selectively be emitted by the excitation source under control of the controller, but for example no third amount of acoustic energy different from the first and second amounts of acoustic energy.

In some variants, only a single type of catheter may be used in such an operational mode. As such, the same catheter may be used in a single procedure to control the excitation source to emit the first amount of acoustic energy in regard to a first segment of a body lumen and the second amount of acoustic energy to a second segment of the same body lumen or another body lumen of the same patient.

The controller may further be configured to control the excitation source to cause the ultrasound transducer to emit the first amount of acoustic energy when the diameter of the segment of the body lumen is determined by the controller, from at least one of a segment diameter estimate determined automatically by the controller and a user specification via a user interface, to be within the lower subrange of the specified range of diameters. Additionally, or in the alternative, the controller may further be configured to control the excitation source to cause the ultrasound transducer to emit the second amount of acoustic energy when the diameter of the segment of the body lumen is determined by the controller, from at least one of a segment diameter estimate determined automatically by the controller and a user specification via a user interface, to be within the upper subrange of the specified range of diameters. The user interface may be configured to display a warning message when the automatically determined estimate of the diameter of the segment of the body lumen is not within the subrange specified via the user interface.

The distal portion of the catheter may further include a balloon within which is located the ultrasound transducer. The balloon may be configured to generally center the ultrasound transducer within the body lumen and to have a fluid circulated through the balloon to cool at least a portion of tissue adjacent to the body lumen within which the ultrasound transducer is positioned.

The balloon may be or comprise a compliant balloon. The compliant balloon may be configured such that when the compliant balloon is partially inflated, such that its diameter is less than a nominal balloon diameter of the compliant balloon, the compliant balloon includes one or more folds. The one or more folds may be configured to at least partially attenuate some of the acoustic energy emitted by the ultrasound transducer. In some implementations, the folds are configured (e.g., when occurring in a predictable manner) such that when the balloon is inserted into a body lumen segment and partially inflated to less than its nominal balloon diameter, a folded surface of the balloon generates additional acoustic reflections compared to when the balloon is inflated to the point that there are no, or less, or smaller folds. The nominal balloon diameter may be within a diameter range of 5.5 mm to 7.5 mm, in particular about 6.5 mm.

In terms of the acoustic patient entry power (i.e., the power “behind” the balloon as seen from the transducer), the compliant balloon may be configured such the attenuation caused by the folds leads to the effect that, at a constant nominal power setting by the controller, the acoustic patient entry power may be somewhat lower for smaller body lumen diameters than larger lumen diameters within the lower subrange (e.g., of diameters from about 3.0 mm to about 4.9 mm), and may be somewhat lower for smaller body lumen diameters than larger lumen diameters within the upper subrange of diameters (e.g., from about 5.0 mm to about 8.0 mm). For example, at a nominal power setting selected within a range from around 25 watts to around 35 watts for the lower subrange (e.g., of ca. 30 watts), the attenuation (e.g., in terms of the acoustic patient entry power) may be around 5% to 15% smaller lower for smaller body lumen diameters than larger lumen diameters within the lower subrange. At a nominal power setting selected within a range from around 31 watts to around 41 watts for the upper subrange (e.g., of about 36 watts), the attenuation (e.g., in terms of the acoustic patient entry power) may be around 2% to 10% smaller lower for smaller body lumen diameters than larger lumen diameters within the upper subrange. In this regard, see also lines 1330 and 1322 in FIG. 13C.

The system may comprise a fluid supply subsystem configured to circulate the fluid through the balloon, and the controller may also be configured to control the fluid supply subsystem. An amount of energy absorbed by tissue surrounding the segment of the body lumen in which the ultrasound transducer is located may depend in part on a flowrate of the fluid circulated through the balloon. In such a case, the controller may be configured to control the flowrate of the fluid circulated through the balloon to be within a flowrate range of about 5 mL/min to about 40 mL/min, in particular about 10 mL/min to about 15 mL/min. The controller may be configured to control the fluid supply system to circulate the fluid through the balloon a predetermined amount of time after the first amount of energy has been emitted by the ultrasound transducer. The predetermined amount of time may be within a range of 2 secs to 12 secs, in particular 5 secs to 9 secs.

A specified intermediate diameter may separate the lower subrange from the upper subrange. The specified intermediate diameter may comprise one of: about 4.5 mm; about 5.0 mm; or about 5.5 mm. The nominal balloon diameter may be selected to be larger than the specified intermediate diameter. For example, the nominal balloon diameter may be selected to be at least 10%, at least 20% or at least 30% larger than the specified intermediate diameter.

A lower end of the specified range of diameters may comprise one of: about 2 mm; about 2.5 mm; or about 3.0 mm. An upper end of the specified range of diameters may comprise one of: about 7.5 mm; about 8.0 mm; or about 8.5 mm.

The second amount of acoustic energy is at least 8%, at least 10%, at least 12% or at least 15% greater than the first amount of acoustic energy. The first amount of acoustic energy may be based on a first acoustic signal having a first acoustic frequency, a first acoustic power and a first duration. The second amount of acoustic energy may be based on an second acoustic signal having a second acoustic frequency, a second acoustic power and a second duration. The second acoustic power may be greater than the first acoustic power. The second acoustic power may be at least 8%, at least 10%, at least 12%, or at least 15% greater than the first acoustic power. The first acoustic frequency may equal the second acoustic frequency. The first duration may equal the second duration. The first and second acoustic powers may be acoustic patient entry powers indicative of an attenuation caused by folds of a compliant balloon and possibly further attenuation effects. Alternatively, the first and second acoustic powers may be nominal acoustic powers as set by the controller (i.e., not yet considering the balloon-related attenuation). A total tolerance range for generating the first and second acoustic powers may be below 15%, below 11% or below 8%.

The range of diameters is at least 4 mm. The controller may be configured to control the excitation source to cause the ultrasound transducer to emit only the two different amounts of acoustic energy.

The first amount of acoustic energy may be based on a first acoustic power within a first range from about 27.5 or 30.0 to about 33.0 watts. The second amount of acoustic energy may be based on a second acoustic power within a second range from about 33.1 to about 39.0 watts. For example, the first amount of acoustic energy is based on a first acoustic power within a first range from about 29.0 or 31.0 to about 33.0 watts and the second amount of acoustic energy is based on a second acoustic power within a second range from about 33.1 to about 38.0 watts. In particular, the first amount of acoustic energy may be based on a first acoustic power of about 30.1 or 32.0 watts and the second amount of acoustic energy is based on a second acoustic power of about 36.0 watts (e.g., 35.8 watts). The first and second acoustic powers may be one of acoustic patient entry powers and nominal acoustic power as set by the controller. See also the illustration in the diagram of FIG. 13C, see line 1330.

Also presented is a method of operating a tissue treatment system, the system comprising a catheter including a distal portion on which is located an ultrasound transducer, wherein the catheter is configured such that at least the distal portion of the catheter is insertable into a segment of a body lumen having a diameter within a specified range of diameters, wherein the range of diameters has a lower subrange and an upper subrange; an excitation source configured to selectively provide energy to the ultrasound transducer of the catheter; and a controller communicatively coupled to the excitation source, the controller configured to control the excitation source to cause the ultrasound transducer to emit two different amounts of acoustic energy, which include a first amount of acoustic energy and a second amount of acoustic energy, which is greater than the first amount of acoustic energy; wherein the method comprises the following steps performed by the controller: determining if a diameter of a segment of a body lumen to be treated is within the lower subrange or the upper subrange; and controlling the excitation source to cause the ultrasound transducer to selectively emit the first amount of acoustic energy if the diameter of the segment of a body lumen to be treated is determined to be within the lower subrange, or to emit the second amount of acoustic energy if the diameter of the segment of the body lumen to be treated is determined to be within the upper subrange. This method may be carried out by any of the two-poser systems described herein.

As noted above, the term “about,” when used to specify a value, means the value+/−10 percent of the value, e.g., “about 3 mm” means 3 mm+/−0.3 mm, and “about 8 mm” means 8 mm +/−0.8 mm.

Although several embodiments and examples are disclosed herein, the present application extends beyond the specifically disclosed embodiments to other alternative embodiments and/or uses and modifications and equivalents thereof. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above, but should be determined only by a fair reading of the claims that follow.

While the present disclosure is susceptible to various modifications, and alternative forms, specific examples thereof have been shown in the drawings and are herein described in detail. It should be understood, however, that the present disclosure is not to be limited to the particular forms or methods disclosed, but, to the contrary, the present disclosure covers all modifications, equivalents, and alternatives falling within the spirit and scope of the various embodiments described and the appended claims. Any methods disclosed herein need not be performed in the order recited.

In the foregoing specification, the present disclosure has been described with reference to specific exemplary embodiments thereof. It will be evident that various modifications may be made thereto without departing from the broader spirit and scope of the present disclosure as set forth in the following claims. The specification and drawings are, accordingly, to be regarded in an illustrative sense rather than a restrictive sense.

The following numbered clauses define further embodiments of the present disclosure.

1. A tissue treatment system, comprising:
a catheter including a distal portion on which is located an ultrasound transducer, wherein the catheter is configured such that at least the distal portion of the catheter is insertable into a segment of a body lumen having a diameter within a specified range of diameters that is at least 4 mm;
an excitation source configured to selectively provide energy to the ultrasound transducer of the catheter; and
a controller communicatively coupled to the excitation source, the controller configured to control the excitation source to cause the ultrasound transducer to emit about a same amount of acoustic energy when the diameter of the segment of the body lumen is within the specified range of diameters that is at least 4 mm.

2. The system of clause 1, wherein:
the specified range of diameters, that is at least 4 mm, includes a lower end of the specified range of diameters and an upper end of the specified range of diameters;
the lower end of the specified range of diameters comprises one of: about 2 mm; about 2.5 mm; or about 3.0 mm; and
the upper end of the specified range of diameters comprises one of: about 7.5 mm; about 8.0 mm; or about 8.5 mm.

3. The system of any one of clause 1 or 2, wherein:
an amount of acoustic energy emitted by the ultrasound transducer is based on an output power of the excitation source that drives the ultrasound transducer, a frequency of an acoustic signal emitted by the ultrasound transducer, and a duration of the acoustic signal emitted by the ultrasound transducer; and
the controller is configured to control the excitation source to cause the ultrasound transducer to emit the same amount of acoustic energy, when the diameter of the segment of the body lumen is within the specified range of diameters, by controlling the excitation source so that the output power is about the same, the frequency of the acoustic signal emitted by the ultrasound transducer is about the same, and the duration of the acoustic signal emitted by the ultrasound transducer is about the same, when the diameter of the segment of the body lumen is within the specified range of diameters that is at least 4 mm.

4. The system of any one of clauses 1 through 3, wherein a distal portion of the catheter further comprises a centering mechanism configured to generally center the ultrasound transducer within the body lumen.

5. The system of any one of clauses 1 through 3, wherein:
the distal portion of the catheter further includes a balloon within which is located the ultrasound transducer; and
the balloon is configured to generally center the ultrasound transducer within the body lumen and to have a fluid circulated through the balloon to cool at least a portion of tissue adjacent to the body lumen within which the ultrasound transducer is positioned.

6. The system of clause 5, further comprising a fluid supply subsystem configured to circulate the fluid through the balloon, and wherein:
the controller is also configured to control the fluid supply subsystem;
an amount of energy absorbed by tissue surrounding the segment of the body lumen in which the ultrasound transducer is located depends in part on a flowrate of the fluid circulated through the balloon; and
the controller is also configured to control the fluid supply subsystem so that the flowrate of the fluid circulated through the balloon is about the same when the diameter of the segment of the body lumen is within the specified range of diameters that is at least 4 mm.

7. The system of clause 6, wherein the flowrate of the fluid circulated through the balloon is within a flowrate range of about 5 mL/min to about 40 mL/min

8. The system of clause 6, wherein the flowrate of the fluid circulated through the balloon is within a flowrate range of about 10 mL/min to about 15 mL/min.

9. The system of any one of clauses 5 through 8, wherein the balloon comprises a compliant balloon.

10. The system of clause 9, wherein the compliant balloon is configured such that when the compliant balloon is partially inflated, such that its diameter is less than a nominal balloon diameter of the compliant balloon, the compliant balloon includes one or more folds.

11. The system of clause 10, wherein the one or more folds comprise at least one of the following: wrinkles; one or more helical folds; or one or more longitudinal folds.

12. The system of any of clause 10 or 11, wherein the one or more folds in the compliant balloon, which is/are present when the compliant balloon is partially inflated such that its diameter is less than the nominal balloon diameter of the compliant balloon, at least partially attenuates some of the acoustic energy emitted by the ultrasound transducer, and thereby reduces an amount of the acoustic energy that passes through compliant balloon when the body lumen in which the compliant balloon is in apposition with has a diameter that is within a smaller diameter subset of the specified range of diameters, compared to when the compliant balloon is inflated such that its diameter is at least the nominal balloon diameter of the compliant balloon and the diameter of the segment of the body lumen in which the compliant balloon is in apposition with is within a larger diameter subset of the specified range of diameters.

13. The system of any one of clauses 9 through 12, wherein:
the compliant balloon has a nominal balloon diameter and a corresponding nominal balloon wall thickness; and
the compliant balloon stretches when the compliant balloon is inflated beyond the nominal balloon diameter, which causes a balloon wall thickness to get thinner than the nominal balloon wall thickness, which results in less attenuation of the acoustic energy emitted by the ultrasound transducer when a diameter of the body lumen in which the compliant balloon is in apposition with is within a larger diameter subset of the specified range of diameters, compared to when the diameter of the segment of the body lumen in which the compliant balloon is in apposition with is within a smaller diameter subset of the specified range of diameters.

14. The system of any one of clauses 9 through 13, wherein the compliant balloon is made from at least one of the following materials: nylon; polyether block amide; or thermoplastic polyurethane.

15. The system of any one of clauses 1 through 14, wherein the about the same amount of acoustic energy, that is emitted by the ultrasound transducer when the diameter of the segment of the body lumen is based on an acoustic entry power within the specified range of diameters, is within a range of acoustic energy from about 30.0 watts to about 39.0 watts.

16. The system of any one of clauses 1 through 14, wherein the about the same amount of acoustic energy, that is emitted by the ultrasound transducer when the diameter of the segment of the body lumen is within the specified range of diameters, is based on an acoustic entry power within a range of acoustic energy from about 32.0 watts to about 36.0 watts.

17. The system of any one of clauses 1 through 14, wherein the about the same amount of acoustic energy, that is emitted by the ultrasound transducer when the diameter of the segment of the body lumen is within the specified range of diameters, is based on an acoustic entry power within a range of acoustic energy from about 33.0 watts to about 35.0 watts.

18. The system of any one of clauses 1 through 17, wherein the body lumen comprises a renal artery.

19. The system of any one of clauses 1 through 17, wherein the body lumen comprises one: a vein; a pulmonary artery; a vascular lumen; a celiac artery; a common hepatic artery; a proper hepatic artery; a gastroduodenal artery; a hepatic artery; a splenic artery; a gastric artery; a blood vessel; a nonvascular lumen; an airway; a sinus; an esophagus; a respiratory lumen; a digestive lumen; a stomach; a duodenum; a jejunum; or a cancer tissue.

20. The system of any one of clauses 1 through 19, wherein the acoustic energy is selected to produce lesions having a depth within a depth range from about 2.5 mm to about 8 mm.

21. The system of any one of clauses 1 through 19, wherein the acoustic energy is selected to produce lesions having a depth within a depth range from about 5.5 mm to about 6.0 mm.

22. The system of any of clauses 10 or 11, wherein the one or more folds in the compliant balloon, which is/are present when the compliant balloon is partially inflated such that its diameter is less than the nominal balloon diameter of the compliant balloon, is/are configured to attenuate more of the acoustic energy emitted by the ultrasound transducer, and thereby is/are configured to allow less of the acoustic energy to pass through the compliant balloon when the compliant balloon is in apposition with a body lumen segment having a diameter that is within a lower diameter subset of the specified range of diameters, compared to when the compliant balloon is in apposition with a body lumen segment that is within a larger diameter subset of the specified range of diameters.

1. A tissue treatment system, comprising:
a catheter including a distal portion on which is located an ultrasound transducer, wherein the catheter is configured such that at least the distal portion of the catheter is insertable into a segment of a body lumen having a diameter within a specified range of diameters, wherein the range of diameters has a lower subrange and an upper subrange;
an excitation source configured to selectively provide energy to the ultrasound transducer of the catheter; and
a controller communicatively coupled to the excitation source, the controller configured to control the excitation source to cause the ultrasound transducer to emit two different amounts of acoustic energy, which include
a first amount of acoustic energy; and
a second amount of acoustic energy, which is greater than the first amount of acoustic energy; wherein
the controller is configured to control the excitation source to cause the ultrasound transducer to emit the first amount of acoustic energy into a body lumen having a diameter within the lower subrange, and the second amount of acoustic energy into a body lumen having a diameter within the upper subrange.

2. The system of claim 1, wherein the controller is further configured to:
automatically estimate a diameter of the segment of the body lumen;
control the excitation source to cause the ultrasound transducer to emit the first amount of acoustic energy when the controller estimated a diameter of the segment of the body lumen to be within the lower subrange of the specified range of diameters; and
control the excitation source to cause the ultrasound transducer to emit the second amount of acoustic energy when the controller estimated a diameter of the segment of the body lumen to be within the upper subrange of the specified range of diameters.

3. The system of claim 2, wherein
the system further comprises a user interface connected to the controller, and the controller is further configured to:
receive a selection from the user interface of one of the lower subrange of the specified range of diameters and the upper subrange of the specified range of diameters, and
control the excitation source to cause the ultrasound transducer to emit the first amount of acoustic energy when the selection is the lower subrange of the specified range of diameters; and
control the excitation source to cause the ultrasound transducer to emit the second amount of acoustic energy when the selection is the upper subrange of the specified range of diameters.

4. The system of claim 1, wherein
the distal portion of the catheter further includes a balloon within which is located the ultrasound transducer; and
the balloon is configured to generally center the ultrasound transducer within the body lumen and to have a fluid circulated through the balloon to cool at least a portion of tissue adjacent to the body lumen within which the ultrasound transducer is positioned.

5. The system of claim 4, wherein
the balloon comprises a compliant balloon, wherein
the compliant balloon is configured such that when the compliant balloon is partially inflated, such that its diameter is less than a nominal balloon diameter of the compliant balloon, the compliant balloon includes one or more folds that at least partially attenuate the acoustic energy emitted by the ultrasound transducer.

6. The system of claim 5, wherein
a specified intermediate diameter separates the lower subrange from the upper subrange, and wherein the nominal balloon diameter is larger than the specified intermediate diameter by an amount selected from the group consisting of at least 10%, at least 20%, and at least 30%.

7. The system of claim 6, wherein
the one or more folds are configured to occur such that when the balloon is inserted into a body lumen segment and partially inflated to less than its nominal balloon diameter, a folded surface of the balloon generates additional acoustic reflections compared to when the balloon is inflated to the point that there are no, or less, or smaller folds.

8. The system of claim 4, further comprising
a fluid supply subsystem configured to circulate the fluid through the balloon, wherein the controller is also configured to control the fluid supply subsystem.

9. The system of claim 8, wherein
an amount of energy absorbed by tissue surrounding the segment of the body lumen in which the ultrasound transducer is located depends in part on a flowrate of the fluid circulated through the balloon, and wherein the controller is configured to control the flowrate of the fluid circulated through the balloon to be within a flowrate range of about 5 mL/min to about 40 mL/min, in particular about 10 mL/min to about 15 mL/min.

10. The system of claim 8, wherein
the controller is configured to control the fluid supply system to circulate the fluid through the balloon, after the first amount of energy has been emitted by the ultrasound transducer, for a predetermined amount of time within a range of 0.5 seconds to 20 seconds.

11. The system of claim 1, wherein:
a lower end of the specified range of diameters is selected from the group consisting of: about 2 mm; about 2.5 mm; and about 3.0 mm; and
an upper end of the specified range of diameters is selected from the group consisting of: about 7.5 mm; about 8.0 mm; and about 8.5 mm.

12. The system of claim 1, wherein
the second amount of acoustic energy is selected from the group consisting of at least 8%, at least 10%, at least 12%, and at least 15% greater than the first amount of acoustic energy.

13. The system of claim 1, wherein
the first amount of acoustic energy is based on a first acoustic signal having a first acoustic frequency, a first acoustic power and a first duration; and
the second amount of acoustic energy is based on an second acoustic signal having a second acoustic frequency, a second acoustic power and a second duration; wherein
the second acoustic power is greater than the first acoustic power.

14. The system of claim 13, wherein
the second acoustic power is selected from the group consisting of at least 8%, at least 10%, at least 12% and at least 15% greater than the first acoustic power.

15. The system of claim 13, wherein
the first acoustic frequency equals the second acoustic frequency.

16. The system of claim 13, wherein
the first duration equals the second duration.

17. The system of claim 13, wherein
a total tolerance range for generating the first and second acoustic powers is selected from the group consisting of: below 15%, below 11% and below 8%.

18. The system of claim 1, wherein:
the first amount of acoustic energy is obtained by a first acoustic power of about 30.1 or 32.0 watts; and
the second amount of acoustic energy is obtained by a second acoustic power of about 36.0 watts.

19. The system of claim 1, wherein the system is configured to produce at least one lesion in tissue surrounding the body lumen in which the ultrasound transducer is positioned, such that a lesion depth of the one or more lesions is within a lesion depth range of about 2 mm to about 10 mm.

20. A method for use with a tissue treatment system having a catheter that includes a distal portion on which is located an ultrasound transducer, the method comprising:
inserting the distal portion of the catheter into a segment of a body lumen having a diameter within a specified range of diameters that is at least 4 mm, such that the ultrasound transducer is located within the segment of the body lumen having the diameter within the specified range of diameters that is at least 4 mm;
receiving input, at a controller associated with the ultrasound transducer, whether the diameter of the segment of the body lumen, within which the ultrasound transducer is located, is within a lower subrange of the specified range of diameters or an upper subrange of the specified range of diameters;
upon receiving input that the diameter of the segment of the body lumen is within the lower subrange of the specified range of diameters, causing the ultrasound transducer to emit a first amount of acoustic energy; and
upon receiving input that the diameter of the segment of the body lumen is within the upper subrange of the specified range of diameters, causing the ultrasound transducer to emit a second amount of acoustic energy, which is greater than the first amount of acoustic energy.

21. The method of claim 20, wherein the receiving input comprises:
accepting an indication, from a user via a user interface connected to the controller, of which one of a plurality of different types of body lumens the ultrasound transducer is located; and
automatically categorizing whether the diameter of the segment of the body lumen, within which the ultrasound transducer is located, is within the lower subrange of the specified range of diameters or the upper subrange of the specified range of diameters, based on the indication accepted via the user interface.

22. The method of claim 20, wherein the receiving input comprises receiving input from the controller, which is configured to automatically determine whether the diameter of the segment of the body lumen, within which the ultrasound transducer is located, is within the lower subrange of the specified range of diameters or the upper subrange of the specified range of diameters.

23. The method of claim 20, wherein the distal portion of the catheter further includes a compliant balloon within which is located the ultrasound transducer, wherein the compliant balloon is configured such that when the compliant balloon is partially inflated, to a diameter less than a nominal balloon diameter of the compliant balloon, the compliant balloon includes one or more folds; the method further comprising:
causing inflation of the compliant balloon so that the compliant balloon is in apposition with a body lumen wall of a segment of the body lumen within which is located the ultrasound transducer;
utilizing the one or more folds in the compliant balloon to at least partially attenuate some of the acoustic energy emitted by the ultrasound transducer, and thereby reduce an amount of the acoustic energy that passes through the compliant balloon when the diameter of the segment of the body lumen in which the compliant balloon is in apposition is within a smaller diameter subset of the specified range of diameters, compared to when the compliant balloon is inflated such that its diameter is at least the nominal balloon diameter of the compliant balloon and the diameter of the segment of the body lumen in which the compliant balloon is in apposition with is within a larger diameter subset of the specified range of diameters.

24. The method of claim 23, wherein the compliant balloon has a balloon wall having a nominal balloon wall thickness; and
wherein the causing inflation comprises causing inflation beyond the nominal balloon diameter; and
wherein the causing inflation beyond the nominal balloon diameter causes the balloon wall to stretch and become thinner than the nominal balloon wall thickness, wherein the thinner balloon wall provides less attenuation of the acoustic energy emitted by the ultrasound transducer than the nominal balloon wall thickness.

25. The method of claim 20, wherein the body lumen is one of a vein; a main renal artery, an accessory renal artery, a renal artery branch, a pulmonary artery; a vascular lumen; a celiac artery; a common hepatic artery; a proper hepatic artery; a gastroduodenal artery; a hepatic artery; a splenic artery; a gastric artery; a blood vessel; a nonvascular lumen; an airway; a sinus; an esophagus; a respiratory lumen; a digestive lumen; a stomach; a duodenum; a jejunum; or a cancer tissue.

26. A tissue treatment system, comprising:
a catheter including a distal portion configured to deliver neuromodulation energy to a segment of a body lumen;
an excitation source configured to selectively provide neuromodulation energy to the catheter; and
a controller communicatively coupled to the excitation source, the controller configured to control the excitation source to cause the catheter to emit about a same amount of neuromodulation energy when a diameter of the segment of the body lumen is within a first specified range of diameters that is at least 4 mm.

27. The system of claim 26, wherein the controller is further configured to control the excitation source to cause the catheter to emit a different amount of neuromodulation energy when the diameter of the segment of the body lumen is within a second specified range of diameters that is at least 1 mm.

28. The system of claim 27, wherein:
the first specified range of diameters, that is at least 4 mm, includes diameters between about 4.1 mm to about 8.0 mm; and
the second specified range of diameters, which is at least 1 mm, includes diameters between about 2.0 mm to about 4.0 mm.

29. The system of claim 26, wherein:
the distal portion configured to deliver neuromodulation energy to a segment of a body lumen comprises an ultrasound transducer, wherein the neuromodulation energy is acoustic energy;
an amount of acoustic energy emitted by the ultrasound transducer is based on an output power of the excitation source that drives the ultrasound transducer, a frequency of an acoustic signal emitted by the ultrasound transducer, and a duration of the acoustic signal emitted by the ultrasound transducer; and
the controller is configured to control the excitation source to cause the ultrasound transducer to emit the same amount of acoustic energy, when the diameter of the segment of the body lumen is within the first specified range of diameters, by controlling the excitation source so that the output power is about the same, the frequency of the acoustic signal emitted by the ultrasound transducer is about the same, and the duration of the acoustic signal emitted by the ultrasound transducer is about the same, when the diameter of the segment of the body lumen is within the first specified range of diameters that is at least 4 mm.

30. The system of claim 29, wherein:
the distal portion of the catheter further includes a balloon within which is located the ultrasound transducer; and
the balloon is configured to generally center the ultrasound transducer within the body lumen and to receive a fluid circulated through the balloon to cool at least a portion of tissue adjacent to the body lumen within which the ultrasound transducer is positioned.

31. The system of claim 30, further comprising a fluid supply subsystem configured to circulate the fluid through the balloon, and wherein:
the controller is also configured to control the fluid supply subsystem;
an amount of energy absorbed by tissue surrounding the segment of the body lumen in which the ultrasound transducer is located depends in part on a flowrate of the fluid circulated through the balloon; and
the controller is also configured to control the fluid supply subsystem so that the flowrate of the fluid circulated through the balloon is about the same when the diameter of the segment of the body lumen is within the specified range of diameters that is at least 4 mm.

32. The system of claim 30, wherein the balloon is a compliant balloon having a Shore D durometer between 50 and 60.
